SEQUENCE LISTING (2 sequences)

SEQ ID NO: 1 - DNA, 20 bp, synthetic construct
  source 1..20: mol_type = other DNA; note = AAVS1 sgRNA sequence
gtcaccaatcctgtccctag

SEQ ID NO: 2 - DNA, 20 bp, synthetic construct
  source 1..20: mol_type = other DNA; note = Universal sgRNA sequence
gggaggcgttcgggccacag

(19) United States
(12) Patent Application Publication
(10) Pub. No.: US 2025/0197826 A1
(43) Pub. Date: Jun. 19, 2025

(54) GENETICALLY ENGINEERED T CELL FOR CELL THERAPY

(71) Applicant: REGENTS OF THE UNIVERSITY OF MINNESOTA, Minneapolis, MN (US)

(72) Inventors: Branden S. Moriarity, Minneapolis, MN (US); Beau R. Webber, Minneapolis, MN (US); Kanut Laoharawee, Minneapolis, MN (US); Evan Kleinboehl, Minneapolis, MN (US)

(21) Appl. No.: 18/847,371

(22) PCT Filed: Mar. 16, 2023
(86) PCT No.: PCT/US23/64604

§ 371 Date: Sep. 16, 2024

Related U.S. Application Data
(60) Provisional application No. 63/320,461, filed on Mar. 16, 2022.

(51) Int. Cl. C12N 9/22; A61K 35/17; C07K 14/705; C12N 15/11; C12N 15/86; C12N 15/90

(52) U.S. Cl. CPC C12N 9/22; A61K 35/17; C07K 14/70596; C12N 15/111; C12N 15/86; C12N 15/902; C12N 2310/20; C12N 2500/32; C12N 2501/2302; C12N 2501/2307; C12N 2501/2315; C12N 2501/24; C12N 2510/00; C12N 2750/14143

(57) ABSTRACT

The present disclosure relates, in general, to a genetically engineered T cell comprising a modified genome expressing a protein of interest, and use thereof in cell therapy.

### CROSS-REFERENCE TO RELATED APPLICATIONS

This application claims the priority benefit of U.S. Provisional Patent Application 63/320,461, filed Mar. 16, 2022, herein incorporated by reference in its entirety.

### INCORPORATION BY REFERENCE OF MATERIAL SUBMITTED ELECTRONICALLY

This application includes a sequence listing submitted electronically, in a file entitled “57363_Seqlisting.xml,” created on Mar. 16, 2023 and having a size of 3,161 bytes, which is incorporated by reference herein.

### FIELD OF THE DISCLOSURE

The present disclosure relates, in general, to a genetically engineered T cell comprising a modified genome expressing a protein of interest and use thereof in cell therapy.

### BACKGROUND

T lymphocytes or T cells are adaptive immune cells derived from lymphoid-lineage progenitors of hematopoietic stem cells in the bone marrow. T cells expressed at least CD3 and CD45 on their surface membrane. They are one of the most abundant leukocytes in the blood and can be easily extracted from the periphery. Upon activation via T-cell receptors (TCRs), T cells can differentiate further into memory T cells that can live for decades.

Genetic modification of autologous T cells has been explored for therapy in cancer and autoimmune disease due to the persistence of treatment and the low risks of rejection by the patient. The CRISPR/Cas9 system provides a method to alter a specific gene of interest by creating a targeted double stranded break (DSB), leading to formation of small insertions or deletions created by semi-random repair via the Non-Homologous End Joining (NHEJ) pathway.

### SUMMARY OF THE DISCLOSURE

The present disclosure provides gene editing systems for targeted insertion of a single gene or multiple gene coding sequences of proteins or enzymes in T cells and use of the engineered T cells as a cell-based gene therapy. It is contemplated that the gene edited T cells are useful as a cell-based protein/enzyme replacement therapies for protein deficiencies or enzymopathies.

Provided herein is a method for genome engineering a T cell or a population of T cells to overexpress a gene of interest comprising introducing into the T cell or T cell population a viral vector, plasmid, nanoplasmid or mini-circle comprising an expression cassette comprising a homology arm (HA), a polynucleotide encoding a gene of interest, a splice acceptor site, a promoter and a targeting site for a nuclease dependent cleavage system targeting molecule, wherein the expression cassette is inserted at a locus targeted by the targeting molecule.

Also contemplated is a method for genome engineering a T cell or a population of T cells to overexpress an endogenous gene comprising introducing into the T cell or T cell population a viral vector, plasmid, nanoplasmid or mini-circle comprising an expression cassette comprising a homology arm(s) (HA), a splice acceptor site, a promoter and a targeting site for a nuclease dependent cleavage system targeting molecule, wherein the expression cassette is inserted upstream of a target gene to be overexpressed.

In various embodiments, the homology arms are between 35 and 1000 nucleotides. In various embodiments, the homology arms are from 50-900 nucleotides, 50-750 nucleotides, 100-600 nucleotides, 100-500 nucleotides, or 200-400 nucleotides. In various embodiments, the homology arms are 35, 40, 45, 50, 55, 60, 65, 70, 75, 80, 90, 100, 125, 150, 175, 200, 250, 300, 350, 400, 450, 500, 550, 600, 650, 700, 750, 800, 850, 900, 950 or 1000 nucleotides.

In various embodiments, the nuclease dependent cleavage system comprises a CRISPR/Cas system, a Cas-CLOVER system, a zinc-finger nuclease (ZFN) system, a transcription activator like effector nuclease (TALEN) system, or a meganuclease system. In various embodiments, the CRISPR/Cas system comprises Cas9, Cas12a, Cas13a or Cas13b.

In various embodiments, the nuclease dependent cleavage system is a CRISPR/Cas system and the targeting molecule is a guide RNA.

In various embodiments, the method further comprises transfecting the T cell or population of T cells with a Cas protein or polynucleotide encoding a Cas protein and guide RNA molecules that direct integration of the expression cassette to a target locus in the T cell genome. In various embodiments, the target locus is an AAVS1 locus or T-cell receptor a constant (TRAC) locus.

In various embodiments, the plasmid, viral vector, nanoplasmid or mini-circle comprises inverted terminal repeats flanking the polynucleotide encoding a gene of interest for transposon delivery. In various embodiments, the plasmid, viral vector, nanoplasmid or mini-circle for transposon delivery comprises a promoter, a gene of interest, a biomarker, a regulatory element and optionally a chimeric intron.

In various embodiments, the method further comprises introducing into the T cell or T cell population a polynucleotide encoding a biomarker molecule useful to enrich for the T cell or a population of T cells. In various embodiments, the biomarker molecule comprises a fragment of CD34 and a fragment of CD20. In various embodiments, the biomarker polynucleotide is on the same expression cassette as the homology arm(s), splice acceptor site, targeting site for a nuclease dependent cleavage system targeting molecule and promoter.

In various embodiments, the viral vector is a lentiviral vector, adenoviral vector, or AAV vector. In various embodiments, the viral vector is selected from the group consisting of a VSVg-pseudotype lentiviral vector, adenoviral vector, AAV6 vector, AAV1 vector or AAV-DJ vector.

In various embodiments, the gene of interest integrates into the T cell genome via homology directed repair (HDR), homology-mediated end joining (HMEJ) or a combination of HDR/HMEJ.

In various embodiments, the introduction of the plasmid, nanoplasmid or mini-circle is by transfection or electroporation. In various embodiments, the introduction of the viral vector is by electroporation. In various embodiments, the viral vector is introduced at a multiplicity of infection (MOI) of 3×10⁵-1×10⁷. In various embodiments, a plasmid, viral vector, nanoplasmid or mini-circle for transposon delivery is electroporated with transposase mRNA and an expression cassette expressing the gene of interest.

In various embodiments, efficiency of introduction is greater than 15%. In various embodiments, the efficiency of introduction is greater than 17%, 20%, 25%, 30%, 35%, 40%, 45%, 50%, 55% or more. In various embodiments, the T cell population has a viability of greater than 60%, 70%, 80%, 90% or more after 3 days. In various embodiments, the T cell population has a viability of greater than 60% after 3 days.

In various embodiments, the T cell or population of T cells is a CD4+ T cell, CD8+ T cell, T cell line, primary T cell, naïve T cell, effector T cell, regulatory T cell, memory T cell, or gamma-delta T cell.

In various embodiments, the viral vector, plasmid, nanoplasmid or mini-circle comprises the promoter next to or near the gene of interest. In various embodiments, the promoter is an exogenous T cell promoter. In various embodiments, the promoter is an MND promoter, a CMV promoter, a CAG promoter, a PGK promoter, a EF1A promoter, or a T cell-specific promoter.

In various embodiments, the gene of interest is a therapeutic gene or encodes a therapeutic protein. In various embodiments, the therapeutic gene encodes an enzyme, a cytokine, a chemokine, a T cell receptor, or a cell surface receptor. In various embodiments, the therapeutic gene encodes an enzyme, a cancer antigen, a cytokine, a chemokine, a T cell receptor, or a cell surface receptor.

In various embodiments the therapeutic protein is an enzyme, a cancer antigen, a cytokine, a chemokine, a T cell receptor, a cell surface receptor, a protein associated with a protein deficiency, or an extracellular membrane protein.

In various embodiments, the gene of interest is a donor polynucleotide that corrects a mutated genotype in a subject.

In various embodiments, the viral vector, plasmid, nanoplasmid or mini-circle further comprises a polynucleotide encoding a T cell receptor or fragment thereof or a chimeric antigen receptor (CAR).

Also provided is a method of making a gene edited T cell or population of T cells, comprising: i) contacting a T cell or population of T cells with a viral vector, plasmid, nanoplasmid or mini-circle comprising an expression cassette comprising a homology arm (HA), a splice acceptor site, a promoter and a targeting site for a nuclease dependent cleavage system targeting molecule, and optionally comprising a polynucleotide encoding a gene of interest; ii) culturing the T cell or population of T cells of i) in a media that promotes expansion of T cells; iii) isolating the T cell or population of T cells of ii) based on identification of a marker expressed only on a T cell or population of T cells carrying the viral vector, plasmid, nanoplasmid or mini-circle; and iv) culturing the isolated cells of iii) in a culture medium to expand the isolated cells expressing the gene of interest.

Also provided is a method of making a gene edited T cell or population of T cells, comprising: i) contacting a T cell or population of T cells with a viral vector, plasmid, nanoplasmid or mini-circle comprising an expression cassette comprising a homology arm (HA), a splice acceptor site, a promoter and a targeting site for transposon delivery, and optionally comprising a polynucleotide encoding a gene of interest; ii) culturing the T cell or population of T cells of i) in a media that promotes expansion of T cells; iii) isolating the T cell or population of T cells of ii) based on identification of a marker expressed only on a T cell or population of T cells carrying the viral vector, plasmid, nanoplasmid or mini-circle; and iv) culturing the isolated cells of iii) in a culture medium to expand the isolated cells expressing the gene of interest.

In various embodiments, the method of making a gene edited T cell or population of T cells further comprises a step of stimulating, proliferating or activating the T cell or population of T cells prior to the contacting step. In various embodiments, stimulating, proliferating or activating the T cell or population of T cells comprises contacting the cell(s) with one or more of IL-2, IL-7, IL-15, IFN-gamma (IFN-γ), or N-acetyl cysteine (NAC).

In various embodiments, the method of making a gene edited T cell or population of T cells produces gene edited T cells with an efficiency of greater than 15%. In various embodiments, the method maintains 60% viability of cells in culture after 3 days.

Also contemplated is a gene edited T cell or population of T cells made by a method described herein. In various embodiments, provided is a gene edited T cell comprising i) a heterologous polynucleotide sequence encoding a gene of interest integrated in the T cell genome at a target location mediated by a nuclease dependent cleavage system, wherein the heterologous polynucleotide sequence is also flanked by portions of a homology arm and expressed via an endogenous promoter; and ii) a heterologous biomarker molecule.

In various embodiments, the gene edited T cell is a CD4+ T cell, CD8+ T cell, T cell line, primary T cell, naïve T cell, effector T cell, regulatory T cell, memory T cell, or gamma-delta T cell.

In various embodiments, the gene edited T cell or population of cells comprises a gene of interest which is a therapeutic gene or encodes a therapeutic protein. In various embodiments, the therapeutic gene encodes an enzyme, a cancer antigen, a cytokine, a chemokine, a T cell receptor, or a cell surface receptor. In various embodiments, the therapeutic gene encodes an enzyme, a cytokine, a chemokine, a T cell receptor, or a cell surface receptor. In various embodiments, the gene of interest is a donor polynucleotide that corrects a mutated genotype in a subject.

Further contemplated is a method of treating a disease or condition in a subject in need thereof comprising administering to the subject a gene edited T cell or population of T cells as described herein. In various embodiments, the disease is an enzymopathy, an infection, or a genetic disorder.

In various embodiments, the disease is an enzymopathy. In various embodiments, the enzymopathy is selected from the group consisting of aspartylglucosaminuria, cholesterol ester storage disease, Wolman disease, metachromatic leukodystrophy, Danon disease, Fabry disease, Farber lipogranulomatosis, Farber disease, fucosidosis, galactosialidosis types I/II, Gaucher disease types I/II/III, globoid cell leukodystrophy, Krabbe disease, glycogen storage disease II, Pompe disease, GM1-gangliosidosis types I/II/III, GM2-gangliosidosis type I, Tay Sachs disease, GM2-gangliosidosis type II, Sandhoff disease, GM2-gangliosidosis, α-mannosidosis types I/II, β-mannosidosis, mucolipidosis type I, sialidosis types I/II, mucolipidosis types II/III, I-cell disease, mucolipidosis type IIIC, pseudo-Hurler polydystrophy, mucopolysaccharidosis type I, mucopolysaccharidosis type II, Hunter syndrome, mucopolysaccharidosis type IIIA, Sanfilippo syndrome type B, Sanfilippo syndrome type C, Sanfilippo syndrome type D, mucopolysaccharidosis type IIIB, mucopolysaccharidosis type IIIC, mucopolysaccharidosis type IIID, mucopolysaccharidosis type IVA, mucopolysaccharidosis type IVB, Morquio syndrome type A, Moriquio syndrome type B, mucopolysaccharidosis type VI, mucopolysaccharidosis type VII, Sly syndrome, mucopolysaccharidosis type IX, multiple sulfatase deficiency, neuronal ceroid lipofuscinosis, CLN1 Batten disease, CLN2 Batten disease, Niemann-Pick disease types A/B, Niemann-Pick disease, Niemann-Pick disease type C1, Niemann-Pick disease type C2, pycnodysostosis, Schindler disease types I/II, and sialic acid storage disease, hemophilia A, hemophilia B, Christmas disease, Factor VII deficiency, spinal muscular atrophy, and epidermolysis bullosa dystrophica.

In various embodiments, the enzymopathy is mucopolysaccharidosis type I (MPS I) and the gene of interest is iduronidase.

In various embodiments, the administration ameliorates one or more symptoms of MPS I. In various embodiments, the one or more symptoms of MPS I are selected from the group consisting of reduction of glycosaminoglycan (GAG) species in tissues and/or urine, increase of IDUA expression in tissues, improved cognition, and reduced vacuolated endothelial cells/foam cells in tissue,

In various embodiments, the genetic disorder is selected from the group consisting of muscular dystrophy, cystic fibrosis, Sickle cell anemia, β-thalassemia, a lysosomal storage disorder, Adenosine Deaminase Deficiency, Severe Combined Immunodeficiency (SCID), Retinitis Pigmentosa, macular degeneration, and Wiskott-Aldrich Syndrome.

In various embodiment, the T cell is a CD4+ T cell, CD8+ T cell, T cell line, primary T cell, naïve T cell, effector T cell, regulatory T cell, memory T cell, or gamma-delta T cell. In various embodiments, the T cell is first isolated from the subject to be treated and then genetically modified according to a method described herein. In various embodiments, the isolated, genetically modified T cell is replaced into the subject from which it was derived.

Also provided by the disclosure is a polynucleotide expression cassette comprising a homology arm (HA), a polynucleotide encoding a gene of interest, a splice acceptor site, a promoter and a targeting site for a nuclease dependent cleavage system targeting molecule, wherein the expression cassette is capable of insertion at a locus targeted by the targeting molecule.

Further contemplated is a polynucleotide expression cassette comprising homology arm(s) (HA), a splice acceptor site, a promoter and a targeting site for a nuclease dependent cleavage system targeting molecule, wherein the expression cassette is capable of insertion upstream of a target gene to be overexpressed.

In various embodiments, the homology arms are between 35 and 1000 nucleotides. In various embodiments, the homology arms are from 50-900 nucleotides, 50-750 nucleotides, 100-600 nucleotides, 100-500 nucleotides, or 200-400 nucleotides. In various embodiments, the homology arms are 35, 40, 45, 50, 55, 60, 65, 70, 75, 80, 90, 100, 125, 150, 175, 200, 250, 300, 350, 400, 450, 500, 550, 600, 650, 700, 750, 800, 850, 900, 950 or 1000 nucleotides.

In various embodiments, the nuclease dependent cleavage system comprises a CRISPR/Cas system, a Cas-CLOVER system, a zinc-finger nuclease (ZFN) system, a transcription activator like effector nuclease (TALEN) system, or a meganuclease system. In various embodiments, the nuclease dependent cleavage system is a CRISPR/Cas system and the targeting molecule is a guide RNA.

In various embodiments, the locus targeted is an AAVS1 locus or T-cell receptor a constant (TRAC) locus.

In various embodiments, the expression cassette further comprises a polynucleotide encoding a biomarker molecule useful to enrich for the T cell or a population of T cells. In various embodiments, the biomarker molecule comprises a fragment of CD34 and a fragment of CD20.

In various embodiments, the expression cassette comprises the promoter next to or near the gene of interest.

In various embodiments, the promoter is an MND promoter, a CMV promoter, a CAG promoter, a PGK promoter, a EF1A promoter, an AAV promoter, or a T cell-specific promoter.

In various embodiments, the expression cassette encodes a gene of interest which is a therapeutic gene or encodes a therapeutic protein. In various embodiments, the therapeutic gene encodes an enzyme, a cancer antigen, a cytokine, a chemokine, a T cell receptor, or a cell surface receptor. In various embodiments, the therapeutic gene encodes an enzyme, a cytokine, a chemokine, a T cell receptor, or a cell surface receptor. In various embodiments, the gene of interest is a donor polynucleotide that corrects a mutated genotype in a subject.

In various embodiments, the expression cassette further comprises a polynucleotide encoding a T cell receptor or fragment thereof or a chimeric antigen receptor.

Also provided is a viral vector, plasmid, nanoplasmid or mini-circle comprising an expression cassette as described herein.

In various embodiments, the viral vector is a lentiviral vector, adenoviral vector, or AAV vector. In various embodiments, the viral vector is selected from the group consisting of a VSVg-pseudotype lentiviral vector, adenoviral vector, AAV6 vector, AAV1 vector or AAV-DJ vector.

Further aspects and advantages will be apparent to those of ordinary skill in the art from a review of the following detailed description, taken in conjunction with the drawings. While the compositions, articles, and methods are susceptible of embodiments in various forms, the description hereafter includes specific embodiments with the understanding that the disclosure is illustrative, and is not intended to limit the invention to the specific embodiments described herein. For the compositions, articles, and methods described herein, optional features, including but not limited to components, compositional ranges thereof, substituents, conditions, and steps, are contemplated to be selected from the various aspects, embodiments, and examples provided herein.

### BRIEF DESCRIPTION OF THE FIGURES

FIG. 1. rAAV vector constructs. A promoterless GFP cassette comprises of splice acceptor (SA) and GFP coding sequence flanked on both sides with homology arms targeting AAVS1 locus was subcloned into rAAV backbone and packaged into a rAAV vector. CRISPR/Cas9 together with rAAV SA-GFP mediated targeted insertion of the GFP into the T-cell genome. Successful integration will allow GFP to express under the regulation of the endogenous AAVS1 promoter.

FIG. 2A-2B. CRISPR/Cas9 with rAAV AAVS1 SA-GFP mediated efficient GFP transgene insertion in the T cell genome. (FIG. 2A). GFP-positive cells were not observed in the sample that received only rAAV Donor template (rAAV vector only). (FIG. 2B) CRISPR/Cas9 mediated up to 68% engineering efficiency indicated by % GFP-positive T cells.

FIG. 3. Experimental Outline for Pilot study. T cells were isolated peripheral blood mononuclear cells from a healthy donor and stimulated for 2 days prior to engineering. The engineered cells were expanded for an additional 7 days before being sorted. Eight million sorted engineered T cells were injected intraperitoneally (IP) into an IDUA deficient mouse. Blood was collected for plasma IDUA for 4 months post engraftment. At the end of the study, the mouse were sacrificed to determine the persistence of T cells and IDUA/GAG in vital organs.

FIG. 4A-4B. CRISPR/Cas9 with rAAV AAVS1 MND-IDUA-RQR9 mediated efficient transgene insertion in T cells. (FIG. 4A) The MND-IDUA-RQR8 cassette is comprised of an MND promoter followed by IDUA-T2A-RQR8 coding sequence and flanked on both sides of the construct with homology arms targeting AAVS1 locus. (FIG. 4B) CRISPR/Cas9 mediated integration in 58% of T cells.

FIG. 5. High levels of IDUA expression was produced and secreted by engineered T cells in culture. More than 400 nmol/hr/mL of IDUA was measured in the media containing engineered T cells while IDUA was not observed in the media containing non-engineered T cells.

FIG. 6. Engineered T cells can be enriched using RQR8-positive sorting strategy. Engineered T cells were enriched using RQR8-positive enrichment strategy with yield 99.6% purity.

FIG. 7. Assessing plasma IDUA over 24 weeks post engraftment. A single IP injection of the purified engineered T cells into an NSG-IDUA deficient mouse showed upregulated IDUA level at week at week 3 and peaked at week 6 post engraftment. Plasma IDUA level of the treated mouse was comparable to or higher than that of the heterozygous mice throughout 24 weeks post engraftment.

FIG. 8. Human T cells were observed in all of the tested vital organs. Human T cells were observed in all tested vital organs such as the heart, lung, liver, spleen, kidney, brain and the bone marrow of the treated IDUA-deficient mice, while human T cells were not observed in the untreated heterozygous mouse.

FIG. 9. Supraphysiological levels of IDUA activity were observed in most of the tested vital organs. Relative IDUA activity in the tissue lysates from organs was measured. Supraphysiological levels of IDUA were observed in the lung, liver, spleen, kidney, spinal cord, brain and bone marrow of the treated mouse when compared to of the heterozygous level. Only 50% of the heterozygous IDUA was observed in the heart of the treated mouse.

FIG. 10. Example plasmid construct for a non-viral T-cell engineering approach. A MND promoter sequence followed by IDUA-T2A-RQR8 coding sequence with homology arms targeting AAVS1 on the pAAV plasmid backbone was used to demonstrate the feasibility of using plasmid for a non-viral approach to engineer T cells.

FIG. 11A-11B. T cells can be non-virally engineered using CRISPR/Cas9 reagents and a donor DNA template plasmid of a transgene. CRISPR/Cas9 and the pAAV AAVS1 MND-IDUA-RQR8 plasmid were co-transfected via electroporation. The control sample was electroporated with PBS only. Flow cytometry analysis showed 0.92% RQR8-positive T cells in the engineered sample, while no RQR8 observed in the control sample. The low engineering efficiency is due to the plasmid is not optimized for T cell engineering.

FIG. 12. Non-viral plasmid engineered T cells express and secrete IDUA. Culture media from samples were used to assessed IDUA expression using IDUA enzymatic activity assay. The engineered T cells (0.92% RQR8-positive cells; Figure) expressed and secreted IDUA in the media higher than the background IDUA level in the control sample.

FIG. 13. Total RQR8 positive Tm cells virally engineered with an IDUA-RQR8 expression cassette day 11 of culture post engineering as measured by flow cytometry.

FIG. 14. Tissue IDUA contents in treated NSG-IDUA deficient mice at 12 weeks post cell injections.

FIG. 15. Pathological tissue GAG contents in treated NSG-IDUA deficient mice at 12 weeks post cell injections.

FIG. 16. Percent of human CD4 positive cells in the organs of treated NSG-IDUA deficient mice 12 weeks post cell injections as measured by flow cytometry.

FIG. 17. Levels plasma IDUA of treated NSG-IDUA deficient mice over 22 weeks

FIG. 18A. Pathological urine GAG content in treated NSG-IDUA deficient mice through 18 weeks post cell injections. FIG. 18B. Creatinine levels in treated NSG-IDUA deficient mice through 18 weeks post cell injections.

FIG. 19. Tissue IDUA content in treated NSG-IDUA deficient mice at 22 weeks post cell injections.

FIG. 20. Percent of human CD45 positive cells in the organs of treated NSG-IDUA deficient mice at 22 weeks post cell injections.

FIG. 21. Effects of administration of engineered cells on neurocognitive ability of control and treated NSG-IDUA deficient mice as measured by time to exit a Barnes maze.

FIG. 22. Level of IDUA activity secreted into the culture medium from engineered human Tm that were isolated from NSG-IDUA deficient mice spleen and bone marrow post 22 week engraftment.

FIGS. 23A-23D. H&E staining of liver tissue after 22 weeks. (FIG. 23A) Heterozygous NSG mice. (FIG. 23B) NSG-IDUA deficient mice. (FIG. 23C-FIG. 23D) Treated NSG-IDUA deficient mice with engineered Tm cell IP injections.

FIGS. 24A-24C. H&E staining of brain tissue after 22 weeks. (FIG. 24A) Heterozygous NSG mice. (FIG. 24B) NSG-IDUA deficient mice. (FIG. 24C) Treated NSG-IDUA deficient mice with engineered Tm cell IP injections.

FIGS. 25A-25B. IDUA immunohistochemistry (IHC) stain of liver. 400×. (FIG. 25A) Untreated MPS I afflicted tissue with foam cell clusters indicated by black boxes. (FIG. 25B) Treated MPS I afflicted tissue. Black arrows show round lymphocytes with moderate IDUA immunopositivity; gray arrows show irregularly shaped Kupffer cells (macrophages) with strong IDUA immunopositivity; inset displays Kupffer cell.

FIG. 26A-26C. IDUA staining of brain tissue after 22 weeks. (FIG. 26A) Heterozygous NSG mice. (FIG. 26B) NSG-IDUA deficient mice. (FIG. 26C) NSG-IDUA deficient mice with engineered Tm cell IP injections.

FIG. 27A-27C. LAMP-1 IHC stain of brain. (FIG. 27A) Heterozygous, healthy tissue. (FIG. 27B) Untreated MPS I afflicted tissue. (FIG. 27C) Treated MPS I afflicted tissue. Black arrows, astrocytes; red arrows, neurons.

FIGS. 28A-28D. CD3 staining of brain tissue after 22 weeks. (FIG. 28A) Heterozygous NSG mice. (FIG. 28B) NSG-IDUA deficient mice. (FIG. 28C-FIG. 28D) NSG-IDUA deficient mice with engineered Tm cell IP injections. Red arrows show nonspecific staining of astrocytes. Black arrows show CD3+ round lymphocytes in leptomeninges.

FIG. 29. Two possible transposon cassettes comprising the IDUA gene and an EGFR reporter.

FIG. 30. EGFR+ Human Bulk T Cells transfected with two IDUA transposon cassettes as measured by flow cytometry.

FIG. 31. Non-viral transposon delivery of an IDUA gene into bulk T Cells results in expression and secretion of IDUA.

FIG. 32. Levels of CD4 and CD8 T cell subsets expressing EGFR from transposon gene delivery

FIG. 33. EGFR+ murine P14 TCRVα2Vβ8 CD8 T cells with transfected with two IDUA transposon cassettes as measured by flow cytometry.

FIG. 34. Non-viral transposon delivery of an IDUA gene into murine P14 TCRVα2Vβ8 CD8 T cells express and secrete IDUA.

FIG. 35. Expression constructs in which the homology arms target the endogenous COL7A1 locus.

FIG. 36. Frequency of successfully targeted γδ T cells as measured by tEGFR expression.

FIG. 37. Relative gene expression of COL7A1 from engineered gamma delta T cells.

### DETAILED DESCRIPTION

Precision genetic modification of primary human T cells has multiple applications in the fields of immunotherapy for cancers, infections, autoimmune diseases, and enzymopathies. Genetic modification of autologous T cells is an attractive avenue for therapy due to the persistence of treatment and the low risks of rejection by the patient. Alternatively, precise genome modifications can be achieved by the introduction of the CRISPR/Cas9 system to induce a targeted DSB along with a DNA template for Homology directed repair (HDR), thereby integrating the DNA template into the host genome. This DNA template can be designed to encode a transgene of interest such as a therapeutic protein/enzyme that can be used as a cell-based gene therapy for a protein/enzyme deficiency.

### Definitions

Unless otherwise defined, all technical and scientific terms used herein have the same meaning as commonly understood by one of ordinary skill in the art to which this invention belongs. The following references provide one of skill with a general definition of many of the terms used in this invention: Singleton et al., DICTIONARY OF MICROBIOLOGY AND MOLECULAR BIOLOGY (3d ed. 2006); THE CAMBRIDGE DICTIONARY OF SCIENCE AND TECHNOLOGY (Walker ed., 1990); THE GLOSSARY OF GENETICS, 5TH ED., R. Rieger et al. (eds.), Springer Verlag (1991); and Hale & Marham, THE COLLINS DICTIONARY OF BIOLOGY (3d, 2005).

Each publication, patent application, patent, and other references cited herein is incorporated by reference in its entirety to the extent that it is not inconsistent with the present disclosure.

It is noted here that as used in this specification and the appended claims, the singular forms “a,” “an,” and “the” include plural reference unless the context clearly dictates otherwise.

“Amplification” refers to any means by which a polynucleotide sequence is copied and thus expanded into a larger number of polynucleotide molecules, e.g., by reverse transcription, polymerase chain reaction, and ligase chain reaction.

“cDNA” refers to a DNA that is complementary or identical to an mRNA, in either single stranded or double stranded form.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand having the same sequence as an mRNA transcribed from that DNA and which are located 5′ to the 5′-end of the RNA transcript are referred to as “upstream sequences”; sequences on the DNA strand having the same sequence as the RNA and which are 3′ to the 3′ end of the coding RNA transcript are referred to as “downstream sequences.”

“Complementary” refers to the topological compatibility or matching together of interacting surfaces of two polynucleotides. Thus, the two molecules can be described as complementary, and furthermore, the contact surface characteristics are complementary to each other. A first polynucleotide is complementary to a second polynucleotide if the nucleotide sequence of the first polynucleotide is identical to the nucleotide sequence of the polynucleotide binding partner of the second polynucleotide. Thus, the polynucleotide whose sequence 5′-TATAC-3′ is complementary to a polynucleotide whose sequence is 5′-GTATA-3′. A nucleotide sequence is “substantially complementary” to a reference nucleotide sequence if the sequence complementary to the subject nucleotide sequence is substantially identical to the reference nucleotide sequence.

“Conservative substitution” refers to the substitution in a polypeptide of an amino acid with a functionally similar amino acid. The following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

The term “fragment” when used in reference to polypeptides refers to polypeptides that are shorter than the full-length polypeptide by virtue of truncation at either the N-terminus or C-terminus of the protein or both, and/or by deletion of an internal portion or region of the protein. Fragments of a polypeptide can be generated by methods known in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Expression control sequence” refers to a nucleotide sequence in a polynucleotide that regulates the expression (transcription and/or translation) of a nucleotide sequence operatively linked thereto. “Operatively linked” refers to a functional relationship between two parts in which the activity of one part (e.g., the ability to regulate transcription) results in an action on the other part (e.g., transcription of the sequence). Expression control sequences can include, for example and without limitation, sequences of promoters (e.g., inducible or constitutive), enhancers, transcription terminators, a start codon (i.e., ATG), splicing signals for introns, and stop codons.

The term “promoter” as used herein refers to a region of DNA that functions to control the transcription of one or more DNA sequences, and that is structurally identified by the presence of a binding site for DNA-dependent RNA-polymerase and of other DNA sequences, which interact to regulate promoter function. A functional expression promoting fragment of a promoter is a shortened or truncated promoter sequence retaining the activity as a promoter. Promoter activity may be measured in any of the assays known in the art e.g. in a reporter assay using Luciferase as reporter gene (Wood, 1991; de Wet et al. (1985), or commercially available.

The term “vector” refers to any carrier of exogenous DNA or RNA that is useful for transferring exogenous DNA to a host cell for replication and/or appropriate expression of the exogenous DNA by the host cell. “Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in vitro expression system. Expression vectors include all those known in the art, such as viral vectors, cosmids, plasmids (e.g., naked or contained in liposomes), nanoplasmids or mini-circles that incorporate the recombinant polynucleotide.

“Nanoplasmid™” refers to a 500 base-pair circular plasmid lacking traditional bacterial genes or antibiotic resistant genes, which in turn reduces cellular toxicity and inflammation of the plasmid-transfected cells, when compared to traditional plasmid. Mini-circles similarly lack bacterial genes.

“Mini-circle” refers to a small circular plasmid derivative of approximately 4 kb that lacks bacterial polynucleotide sequences. Mini-circles can be non-replicating and lack an origin of replication, or can be modified to comprise a non-bacterial replication element (e.g., a S/MAR element).

“Viral vector” refers to a vector that uses a viral backbone for carrying a polynucleotide expression cassette. Viral vectors include lentiviral vectors, adenoviral vectors or adeno-associated vectors (AAV).

“Expression cassette” or “cassette” refers to a component of vector or plasmid DNA that controls expression of a gene or protein, and may be interchangeable and easily inserted or removed from a vector. Expression cassettes often comprise a promoter sequence, an open reading frame, and a 3′ untranslated region that contains a polyadenylation site. A “therapeutic expression cassette” or “therapeutic cassette” refers to an expression cassette expressing a therapeutic protein for use in treating disease.

An “enhancer region” refers to a region of DNA that functions to increase the transcription of one or more genes. More specifically, the term “enhancer”, as used herein, is a DNA regulatory element that enhances, augments, improves, or ameliorates expression of a gene irrespective of its location and orientation. It is contemplated that an enhancer may enhance expression of more than one promoter.

A “homology arm” refers to a polynucleotide sequence at the 5′ region and at the 3′ region immediately flanking a DNA sequence of interest in an expression cassette that possess homology to a selected insertion site in genomic DNA/RNA for the purpose of carrying out homologous recombination.

“Transposon delivery” refers to use of transposon sequences and transposase enzyme for site-specific delivery of DNA to a cell genome. A DNA transposase makes a staggered cut at the target site producing sticky ends, cuts out the DNA transposon and ligates it into the target site. Exemplary transposons include, but are not limited to, Tn5, Tn3, Tn10, Sleeping Beauty, piggyBac, and Tol2.

“Polynucleotide” refers to a polymer composed of nucleotide units. Polynucleotides include naturally occurring nucleic acids, such as deoxyribonucleic acid (“DNA”), including cDNA, and ribonucleic acid (“RNA”) as well as nucleic acid analogs. Nucleic acid analogs include those which include non-naturally occurring bases, nucleotides that engage in linkages with other nucleotides other than the naturally occurring phosphodiester bond or which include bases attached through linkages other than phosphodiester bonds. Thus, nucleotide analogs include, for example and without limitation, phosphorothioates, phosphorodithioates, phosphorotriesters, phosphoramidates, boranophosphates, methylphosphonates, chiral-methyl phosphonates, 2-O-methyl ribonucleotides, peptide-nucleic acids (PNAs), and the like. Such polynucleotides can be synthesized, for example, using an automated DNA synthesizer. The term “nucleic acid” typically refers to large polynucleotides. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer. The term “protein” typically refers to large polypeptides. The term “peptide” typically refers to short polypeptides. Conventional notation is used herein to portray polypeptide sequences: the left-hand end of a polypeptide sequence is the amino-terminus; the right-hand end of a polypeptide sequence is the carboxyl-terminus.

“Recombinant polynucleotide” refers to a polynucleotide having sequences that are not naturally joined together. An amplified or assembled recombinant polynucleotide may be included in a suitable vector, and the vector can be used to transform a suitable host cell. A host cell that comprises the recombinant polynucleotide is referred to as a “recombinant host cell.” The gene is then expressed in the recombinant host cell to produce, e.g., a “recombinant polypeptide.” A recombinant polynucleotide may serve a non-coding function (e.g., promoter, origin of replication, ribosome-binding site, etc.) as well. Recombinant protein refers to a protein encoded by a recombinant polynucleotide.

“Substantially pure” or “isolated” means an object species is the predominant species present (i.e., on a molar basis, more abundant than any other individual macromolecular species in the composition), and a substantially purified fraction is a composition wherein the object species comprises at least about 50% (on a molar basis) of all macromolecular species present. Generally, a substantially pure composition means that about 80% to 90% or more of the macromolecular species present in the composition is the purified species of interest. The object species is purified to essential homogeneity (contaminant species cannot be detected in the composition by conventional detection methods) if the composition consists essentially of a single macromolecular species. Solvent species, small molecules (<500 Daltons), stabilizers (e.g., BSA), and elemental ion species are not considered macromolecular species for purposes of this definition. In various embodiments, recombinant proteins of the disclosure are substantially pure or isolated with respect to the macromolecular starting materials used in their synthesis. In various embodiments, the pharmaceutical composition of the disclosure comprises a substantially purified or isolated therapeutic protein admixed with one or more pharmaceutically acceptable carriers, diluents or excipients.

The term “specifically binds” is “antigen specific”, is “specific for”, “selective binding agent”, “specific binding agent”, “antigen target” or is “immunoreactive” with an antigen refers to a T cell receptor or polypeptide that binds a target antigen with greater affinity than other antigens of related proteins.

The term “endogenous” refers to a protein, polynucleotide, or other molecule that is naturally found in, or expressed by, a subject, e.g., a cell, organ, or tissue. The term “exogenous” refers to a protein, polynucleotide, or other molecule that is not naturally found in a subject, e.g., a cell, organ, or tissue.

The term “genetically engineered” as used herein refers to a polynucleotide or polypeptide sequence that has been modified from its naturally-occurring sequence, e.g., by insertion, deletion or polynucleotide or amino acid substitution/modification, using recombinant DNA expression techniques to produce a polypeptide or polynucleotide sequence that differs from the previously unmodified sequence.

The term “nuclease dependent cleavage system” as used herein refers to gene editing techniques that employ DNA or RNA dependent nucleases to cleave target DNA or RNA, respectively, and molecules or guides that direct the nuclease to the target DNA/RNA to be cleaved. Examples of nuclease dependent cleavage systems include CRISPR/Cas systems, Cas-CLOVER systems, zinc-finger nuclease (ZFN) systems, transcription activator like effector nuclease (TALEN) systems, or meganuclease systems.

“Homozygous” for the donor polynucleotide as used herein refers to the result of the genetic modification in which both alleles of the modified gene express the donor polynucleotide. “Heterozygous” for the donor polynucleotide as used herein refers to the result of the genetic modification in which only one of the alleles of the gene express the donor polynucleotide.

### T Cells

Upon activation via T-cell receptors (TCRs), T cells can differentiate into memory T cells that can live for decades. The abundance of T cells and the ability to differentiate into long-lived cells make T cells attractive candidates for use as a cell-based gene therapy.

A “T cell receptor” or “TCR” refers to a multisubunit protein comprising either α and β chains (TCR αβ) which together bind to a peptide-MHC ligand, or γ and δ subunits (TCRγδ). Each chain is composed of two extracellular domains comprising variable (V) region and a constant (C) region. The variable region binds to the peptide/MHC complex. The variable domain of both the TCR α-chain and β-chain each have three hypervariable or complementarity-determining regions (CDRs). The TCRαβ is complexed with CD3 and other proteins in the T cell to mediate signaling through the T cell receptor.

It is contemplated that TCR specific for certain infectious microbes, such as virus or bacteria, or other known binding antigens can be engineered as described herein. Exemplary TCRs include, but are not limited to TCR specific for Hepatitis B virus (HBV), human immunodeficiency virus (HIV), Epstein-Barr virus (EBV), Influenza A, coronavirus Covid19, cytomegalovirus (CMV), Yellow Fever vaccine, Salmonella typhi, Tetanus, Diphtheria, and Tuberculosis.

In various embodiments, the T cell can be a CD4+ cell. In various embodiments, the T cell can be a CD8+ cell. In various embodiments, the T cell can be a primary T cell. As used herein, a “primary T cell” is a non-immortalized T cell. In various embodiments, a “primary T cell” is a T cell that is freshly isolated. In various embodiments, the T cell can be derived from blood or serum.

In various embodiments, a “primary T cell” is a T cell that has undergone up to 5 replications or divisions after being isolated, up to 10 replications or divisions after being isolated, up to 15 replications or divisions after being isolated, up to 20 replications or divisions after being isolated, up to 25 replications or divisions after being isolated, up to 30 replications or divisions after being isolated, up to 35 replications or divisions after being isolated, or up to 40 replications or divisions after being isolated.

In various embodiments, a “primary T cell” is a T cell that has undergone up to 5 replications or divisions after being derived, up to 10 replications or divisions after being derived, up to 15 replications or divisions after being derived, up to 20 replications or divisions after being derived, up to 25 replications or divisions after being derived, up to 30 replications or divisions after being derived, up to 35 replications or divisions after being derived, or up to 40 replications or divisions after being derived.

In various embodiments, the primary T cell is a non-clonal cell. In various embodiments, the primary T cell is a proliferating cell. In various embodiments, the T cell is cultured in the presence of IL-2 or other T cell growth medium.

In various embodiments, the T cell is a naïve T cell. In various embodiments, the T cell is an effector T cell. In various embodiments, the T cell is a memory T cell. In various embodiments, a “memory T cell” expresses CD45RO. In various embodiments, the T cell is an activated memory T cell. In various embodiments, the T cell is a resident memory T cell. In various embodiments, the T cell is a regulatory T cell. In various embodiments, the regulatory T cell is FOXP3⁺ or FOXP3⁻. In various embodiments, the T cell is a gamma-delta T cell.

In various embodiments, the T cell is a mammalian cell. In various embodiments, the T cell is a human cell. In various embodiments, the T cell is a mouse cell.

A T cell is “gene edited” if the T cell includes a modification to its genome compared to a non-gene edited T cell. In some embodiments, a non-gene edited T cell is a wild-type T cell. In some embodiments, a non-gene edited T cell is a freshly isolated T cell.

In various embodiments, the gene edited T cell includes a modification of a noncoding region of the genome and/or a coding region of the genome (e.g., a gene). In various embodiments, the noncoding region of the genome can include a sequence for a small, regulatory noncoding RNA, including, for example, a microRNA (miRNA). In various embodiments, the noncoding region of the genome is preferably involved in regulating the function, activation, and/or survival of the T cell.

In various embodiments, a portion of genomic information and/or a gene can be deleted. In various embodiments, a portion of genomic information and/or a gene can be added. In various embodiments, the genomic information and/or the gene that is added is exogenous. In various embodiments, “exogenous” genomic information or an “exogenous” gene can be genomic information or a gene from a non-T cell. In various embodiments, “exogenous” genomic information or an “exogenous” gene can be an additional copy of genomic information or a gene already present in the T cell. In various embodiments, “exogenous” genomic information or an “exogenous” gene can be genomic information or a gene from a cell of another species than the T cell being modified. In various embodiments, “exogenous” genomic information or an “exogenous” gene can be artificially generated including, for example, a nucleic acid encoding a chimeric antigen receptor. In various embodiments, a portion of genomic information and/or a gene can be altered, for example, by a point mutation.

In various embodiments, a gene edited T cell includes a modification that alters expression or activity of the gene edited T cell relative to a non-gene edited T cell. For example, in various embodiments, the gene edited T cell may include an expression cassette or therapeutic cassette, as described herein.

In various embodiments, a gene edited primary T cell includes a modification of a nucleic acid encoding the endogenous T cell receptor (TCR). In various embodiments, the modification results in a modification of the expression of the endogenous TCR. For example, expression of the endogenous TCR may be abrogated relative to a non-gene edited primary T cell. In some embodiments, the expression of the endogenous TCR may be enhanced relative to a non-gene edited primary T cell.

In various embodiments, a gene edited T cell includes a modification of a nucleic acid encoding a cytokine. The cytokine can include, for example, IL-2, IL-4, IL-7, IL-12, IL-13, IL-15, IL-17, IL-21, IL-32, IL-33, IFN-gamma, or combinations thereof.

In various embodiments, a gene edited T cell includes a nucleic acid encoding a chemokine receptor. The chemokine receptor includes, CXCR1, CXCR2, CXCR3, CXCR4, CXCR5, CXCR6, CCR1, CCR2, CCR3, CCR4, CCR5, CCR6, CCR7, CCR8, CCR9, CCR10, or CCR11.

### Vectors

Exemplary expression vectors include, but are not limited to, viral vectors, plasmids, nanoplasmids or mini-circles.

Viral vectors include, but are not limited to the following: viral vectors based on vaccinia virus; poliovirus; adenovirus (see, e.g., Li et al., Invest Opthalmol Vis Sci 35:2543 2549 (1994); Borras et al., Gene Ther 6:515 524 (1999); Li and Davidson, PNAS 92:7700-7704 (1995); Sakamoto et al., H Gene Ther 5:1088-1097 (1999); WO 94/12649, WO 93/03769; WO 93/19191; WO 94/28938; WO 95/11984 and WO 95/00655); adeno-associated virus (see, e.g., Ali et al., Hum Gene Ther 9:81 86 (1998), Flanner et al., PNAS 94:6916-6921 (1997); Bennett et al., Invest Opthalmol Vis Sci 38:2857-2863 (1997); Jomary et al., Gene Ther 4:683-690 (1997), Rolling et al., Hum Gene Ther 10:641-648, (1999); Ali et al., Hum Mol Genet 5:591-594 (1996); Srivastava in WO 93/09239, Samulski et al., J. Vir. 63:3822-3828 (1989); Mendelson et al., Virol. 166:154-165 (1988); and Flotte et al., PNAS 90:10613-10617 (1993)); SV40; herpes simplex virus; human immunodeficiency virus (see, e.g., Miyoshi et al., PNAS 94:10319-23 (1997); Takahashi et al., J Virol 73:7812-7816 (1999)); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus).

Recombinant adeno-associated viral vectors (rAAV) are small DNA viruses, with a packaging capacity of 4.7 kilobase (Kb), that have been used extensively as a vehicle for a DNA template delivery for CRISPR/Cas9 mediated transgene insertion in immune cells, including T cells^{1,2}. The method for genome editing described here can be used to insert a promoter or splice acceptor to drive expression of a transgene encoding a secreted protein or enzyme using either an rAAV or a plasmid as an HDR or HMEJ DNA donor template.

Adenoviral vectors are non-enveloped double-stranded DNA vectors that can be rendered replication-deficient by deletion of the E1 region of the viral genome. Typical adenoviral vectors have a packaging capacity of up to 7.5 kbp. Adenoviral vectors are commonly used in gene therapy clinical trials. Useful adenoviral vectors are described in Wold et al., (Curr Gene Ther. 2013 December; 13(6): 421-433), hereby incorporated by reference.

Lentiviral vectors are enveloped retroviruses with single stranded RNA genomes. Lentivirus is capable of infecting dividing and non-dividing cells. Lentivirus have a genome capacity of approximately 10 kb.

Plasmids contemplated herein include, e.g., naked plasmids or contained in liposomes or another delivery vehicle. A Nanoplasmid™” refers to a 500 base-pair circular plasmid lacking traditional bacterial genes or antibiotic resistant genes. The nanoplasmid construct reduces cellular toxicity and inflammation of the plasmid-transfected cells, when compared to traditional plasmid.

Mini-circles similarly lack bacterial genes and are small circular plasmid derivatives of approximately 4 kb that lack bacterial polynucleotide sequences. Mini-circles can be non-replicating and lack an origin of replication, or can be modified to comprise a non-bacterial replication element (e.g., a S/MAR element).

Vectors for transposon delivery of genetic information are also contemplated. The vectors comprise inverted terminal repeats flanking the polynucleotide encoding a gene of interest for transposon delivery. The vector for transposon delivery comprises a promoter, a gene of interest, a biomarker, a regulatory element and optionally a chimeric intron. In various embodiments, the transposon is selected from the group consisting of Tn5, Tn3, Tn10, Sleeping Beauty, piggyBac, and Tol2.

### Genome Engineering

CRISPR/Cas and other nuclease-based gene editing systems open a new avenue to altering a gene of interest by creating double stranded breaks (DSB), leading to formation of small insertions or deletions created by semi-random repair via the Non-Homologous End Joining (NHEJ) pathway. Alternatively, precise genome modifications can be achieved by the introduction of CRISPR/Cas9 to induce a DSB along with a DNA template for Homology directed repair (HDR). A DNA template can be designed to encode a transgene of interest such as for T cell receptor (TCR) or encode a therapeutic protein/enzyme that can be used for cancer immunotherapy or protein/enzyme deficiency, respectively.

In various embodiments, the method includes a technique to introduce a protein or nucleic acid into the T cell or population of T cells. Any suitable method of introducing a protein or nucleic acid may be used. In various embodiments, the method includes electroporation of a T cell or population of T cells to introduce genetic material including, for example, DNA, RNA, and/or mRNA. As used herein, electroporation may include nucleofection. Because plasmid DNA can be toxic to T cells, in some embodiments, mRNA or protein based approaches of genome editing are used. In various embodiments, a technique to introduce a protein or nucleic acid can include introducing a protein or nucleic acid via electroporation; microinjection; exosomes; liposomes; biolistics; jet injection; hydrodynamic injection; ultrasound; magnetic field-mediated gene transfer; electric pulse-mediated gene transfer; use of nanoparticles including, for example, lipid-based nanoparticles; incubation with an endosomolytic agent; use of cell-penetrating peptides; etc. In various embodiments, the method includes electroporation of a T cell using a NEON transfection system, Lonza transfection system, or MaxCyte transfection system.

In various embodiments, the method includes editing a gene. Editing a gene can include introducing one or more copies of the gene, altering the gene, deleting the gene, upregulating expression of the gene, downregulating expression of the gene, mutating the gene, methylating the gene, demethylating the gene, acetylating the gene, and/or deacetylating the gene. Mutating the gene can include introducing activing mutations, introducing inactivating and/or inhibitory mutations, and/or introducing point mutations.

In various embodiments, the method includes inducing double stranded breaks in the genome of the T cell. Double stranded breaks may be introduced using a nuclease dependent cleavage systems including, for example, a transcription activator-like effector nucleases (TALEN), a zinc finger nuclease (ZFN), a CRISPR-associated nuclease, etc.

Double stranded breaks may also be introduced using a transposase delivery system, or a nuclease dependent cleavage system including, for example, a transcription activator-like effector nucleases (TALEN), a zinc finger nuclease (ZFN), a CRISPR-associated nuclease, etc.

If a CRISPR/Cas system is used, it includes use of a guide RNA (gRNA) or DNA (gDNA) targeting molecule. The gRNA target or gDNA target can include any suitable target. In various embodiments, the target includes a portion of the T cell genome including, for example, a gene or a portion of a gene.

The disclosure herein provides a method for genome engineering a T cell or a population of T cells to overexpress a gene of interest comprising introducing into the T cell or T cell population a viral vector, plasmid, nanoplasmid or mini-circle comprising an expression cassette comprising a homology arm (HA), a polynucleotide encoding a gene of interest, a splice acceptor site, a promoter and a targeting site for a nuclease dependent cleavage system targeting molecule, wherein the expression cassette is inserted at a locus targeted by the targeting molecule.

The disclosure herein provides a method for genome engineering a T cell or a population of T cells to overexpress a gene of interest comprising introducing into the T cell or T cell population a viral vector, plasmid, nanoplasmid or mini-circle comprising an expression cassette comprising a homology arm (HA), a polynucleotide encoding a gene of interest, a splice acceptor site, a promoter and a targeting site for transposon delivery of the gene of interest, wherein the expression cassette is inserted at a locus targeted by the targeting molecule.

Also provided is a method for genome engineering a T cell or a population of T cells to overexpress an endogenous gene comprising introducing into the T cell or T cell population a viral vector, plasmid, nanoplasmid or mini-circle comprising an expression cassette comprising a homology arm(s) (HA), a splice acceptor site, a promoter and a targeting site for a nuclease dependent cleavage system targeting molecule, wherein the expression cassette is inserted upstream of a target gene to be overexpressed.

Also provided is a method for genome engineering a T cell or a population of T cells to overexpress an endogenous gene comprising introducing into the T cell or T cell population a viral vector, plasmid, nanoplasmid or mini-circle comprising an expression cassette comprising a homology arm(s) (HA), a splice acceptor site, a promoter and a targeting site for transposon delivery of a gene of interest, wherein the expression cassette is inserted upstream of a target gene to be overexpressed.

In some embodiments, where transfection may be used to deliver a CRISPR/Cas9 system, the gRNA may preferably include a chemically modified gRNA. In some embodiments, the chemical modification to the gRNA preferably decreases a cell's ability to degrade the RNA. In some embodiments, a chemically modified gRNA includes one or more of the following modifications: 2′-fluoro (2′-F), 2′-O-methyl (2′-O-Me), S-constrained ethyl (cEt), 2′-O-methyl (M), 2′-O-methyl-3′-phosphorothioate (MS), and/or 2′-O-methyl-3′-thiophosphonoacetate (MSP). In some embodiments, the chemically modified gRNA can include a gRNA and/or a chemical modification described in Hendel et al, Nature Biotechnology, 2015, 33(9):985-989 or Rahdar et al., PNAS, 2015, 112(51):E7110-7.

Introduction of the plasmid, nanonplasmid or mini-circle into a cell can be performed by transfection or electroporation.

Introduction of a viral vector is by electroporation. In various embodiments, the viral vector is introduced at a multiplicity of infection (MOI) of 3×10⁵-1×10⁷. In various embodiments, the MOI is between 5×10⁵-1×10⁷, between 5×10⁵-5×10⁶, between 5×10⁵-1×10⁶ or between 3×10⁵-1×10⁶.

A vector for transposon delivery is electroporated with transposase mRNA and an expression cassette for transposon delivery expressing the gene of interest.

It is contemplated that the present method provides efficient transfer of the gene of interest and provides improved viability of the T cells after genome modification. For example, the efficiency of transfer of the gene of interest, or overexpression of an endogenous polynucleotide is 10%, 15%, 20%, 25%, 30%, 35%, 40%, 45%, 50%, 55%, 60%, 65%, 70%, 75% or more. In various embodiments, the T cell population has a viability of greater than 60%, 70%, 80%, 90% or more after 3 days. In various embodiments, the T cell population has a viability of greater than 60% after 3 days.

In various embodiments, the method includes selecting a gene edited T cell. In various embodiments, the selection is performed after editing a gene. A T cell can, in various embodiments, be selected using one or more of the following methods: flow sorting (including, for example, for cell surface marker expression); magnetic bead separation (including, for example, targeting a cell-surface marker); transient drug resistance gene expression (including, for example, antibiotic resistance).

In various embodiments, the method includes expanding a gene edited T cell. In various embodiments, the expansion can be performed after selecting the gene edited T cell. In various embodiments, a T cell can be expanded by co-incubation with an antigen recognized by the T cell receptor or a cell expressing an antigen recognized by the T cell receptor. In various embodiments, a T cell can be expanded (e.g., stimulating, proliferating or activating) by co-incubation with a cytokine or ligand including, for example, contacting the cell(s) with one or more of interleukin-2 (IL-2), interleukin-7 (IL-7), interleukin-15 (IL-15), interferon-gamma (IFN-gamma, IFN-γ), or N-acetyl cysteine (NAC).

In various embodiments, the T cell can be stimulated for at least 18 hours, at least 1 day, at least 36 hours, at least 2 days, at least 3 days, at least 4 days, at least 5 days, at least 6 days, or at least 7 days. In some embodiments, the T cell can be simulated for up to 1 day, up to 2 days, up to 3 days, up to 4 days, up to 5 days, up to 6 days, up to 7 days, up to 8 days, up to 9 days, up to 10 days, up to 12 days, up to 14 days, up to 3 weeks, up to 4 weeks, or up to two months. In some embodiments, the T cell is preferably stimulated for 14 days.

### Nuclease Dependent Cleavage Systems

Zinc-finger nucleases (ZFNs) and Transcription activator-like effector nucleases (TALENs) are customizable DNA-binding proteins that comprise DNA-modifying enzymes. Both can be designed and targeted to specific sequences in a variety of organisms (Esvelt and Wang, Mol Syst Biol. (2013) 9: 641). ZFNs and TALENs are useful to introduce a broad range of genetic modifications by inducing DNA double-strand breaks that stimulate error-prone non-homologous end joining (NHEJ) or homology-directed repair (HDR) at specific genomic locations. These DNA-binding modules can be combined with numerous effector domains to affect genomic structure and function, including nucleases, transcriptional activators and repressors, recombinases, transposases, DNA and histone methyltransferases, and histone acetyltransferases. Thus, the ability to execute genetic alterations depends largely on the DNA-binding specificity and affinity of designed zinc finger and TALEN proteins (Gaj et al., Trends in Biotechnology, (2013) 31(7):397-405). The following U.S. granted patents, incorporated by reference, describe the use of ZFNs and TALENs in mammalian cells, U.S. Pat. Nos. 8,685,737 and 8,697,853.

CRISPR-Cas (clustered regularly interspaced short palindromic repeats-CRISPR associated) is an RNA-mediated adaptive immune system found in bacteria and archaea, which provides adaptive immunity against foreign nucleic acids (Wiedenheft et al., Nature (2012) 482:331-8; Jinek et al., Science (2012) 337:816-21). Recent studies have shown that the biological components of this system can be used to modify to the genome of mammalian cells. CRISPR-Cas systems are generally defined by a genomic locus called the CRISPR array, a series of 20-50 base-pair (bp) direct repeats separated by unique “spacers” of similar length and preceded by an AT-rich “leader” sequence (Wright et al., Cell (2016) 164:29-44).

Three types of CRISPR/Cas systems exist, type I, II and III. The Type II CRISPR-Cas systems require a single protein, e.g., Cas9, to catalyze DNA cleavage (Sapranauskas et al., Nucleic Acids Res. (2011) 39(21): 9275-9282). Cas9 serves as an RNA-guided DNA endonuclease. Cas9 generates blunt double-strand breaks (DSBs) at sites defined by a 20-nucleotide guide sequence contained within an associated CRISPR RNA (crRNA) transcript. Cas9 requires both the guide crRNA and a trans-activating crRNA (tracrRNA) that is partially complementary to the crRNA for site-specific DNA recognition and cleavage (Deltcheva et al., Nature (2011)4 71(7340):602-7; Jinek et al., Science (2012) 337:816-21).

The crRNA:tracrRNA complex can be synthesized as two separate molecules or as a single transcript (single-guide RNA or sgRNA) encompassing the features required for both Cas9 binding and DNA target site recognition. Using sgRNA, Cas from bacterial species, such as S. pyogenes, can be programmed to cleave double-stranded DNA at any site defined by the guide RNA sequence and including a protospacer-adjacent (PAM) motif (Sapranauskas et al., Nucleic Acids Res. (2011) 39(21): 9275-9282; Jinek et al., Science (2012) 337:816-21). The DSBs result in either non-homologous end-joining (NHEJ), which is error-prone and conducive to frameshift mutations that knock out gene alleles, or homology-directed repair (HDR), which can be exploited with the use of an exogenously introduced double-strand or single-strand DNA repair template to knock in or correct a mutation in the genome. Therefore, in the presence of a homologous repair donor, the CRISPR/Cas9 system may be used to generate precise and defined modifications and insertions at a targeted locus through the HDR process. In the absence of a homologous repair donor, single DSBs generated by CRISPR/Cas9 are repaired through the error-prone NHEJ, which results in insertion or deletion (indel) mutations.

Other publications describing the CRISPR systems and Cas9, include the following Cong et al. Science (2013) 339:819-23; Jinek et al., eLife 2013; 2:e00471. (2013) 2:e00471; Lei et al. Cell (2013) 152: 1173-1183; Gilbert et al. Cell (2013) 154:442-51; Lei et al. eLife (2014) 3:e04766; Perez-Pinela et al. Nat Methods (2013) 10: 973-976; Maider et al. Nature Methods (2013) 10, 977-979 which are incorporated by reference. The following U.S. and international patents and patent applications describe the methods of use of CRISPR, 8,697,359; 8,771,945; 8,795,965; 8,865,406; 8,871,445; 8,889,356; 8,895,308; 8,906,616; 8,932,814; 8,945,839; 8,993,233; 8,999,641; 2014/0068797; and WO 2014/197568, each of which is incorporated by reference in their entirety.

The CRISPR related protein, Cas9, can be from any number of species including, but not limited to, Streptococcus pyogenes, Staphylococcus aureus, Listeria innocua, and Streptococcus thermophilus.

Additional Cas proteins known in the art are contemplated for use in the methods, including Cas12a (Cpf1) and Cas 13a/Cas13b (56). See also Yan et al., Cell Biology and Toxicology 35:489-492 (2019).

Cas-CLOVER™ systems are recently designed gene editing systems that utilize the Clo51 nuclease instead of the CRISPR protein. Cas-CLOVER™ comprises a nuclease-inactivated Cas9 protein fused to the Clo51 endonuclease. Cas-CLOVER uses two guide RNAs as well as a nuclease activity that requires dimerization of subunits associated with each guide RNA to provide target specificity.

In one embodiment, the methods use a CRISPR-Cas system and one or more guide RNAs, repair templates and HDR to insert nucleotide bases into the genome of a T cell locus. In various embodiments, the T cell locus is the (TCR) alpha chain (TRAC) locus or AAVS1 locus.

### Nucleic Acid Molecules

Nucleic acids of the disclosure can be cloned into a vector, such as a viral vector, plasmid, nanoplasmid or mini-circle, into which another genetic sequence or element (either DNA or RNA) may be inserted so as to bring about the replication of the attached sequence or element. In various embodiments, the expression vector contains a constitutively active promoter segment (such as but not limited to CMV, SV40, Elongation Factor or LTR sequences) or an inducible promoter sequence such as the steroid inducible pIND vector (Invitrogen), where the expression of the nucleic acid can be regulated. Expression vectors of the disclosure may further comprise regulatory sequences. The vector can be introduced into a cell by transfection, for example.

A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired. For instance, in some embodiments, signal peptide sequences may be appended/fused to the amino terminus of any of the donor polynucleotide, CRISPR-Cas or other nuclease-dependent cleavage system described herein.

A vector may also comprise a nucleic acid comprising a promoter, a coding sequence of a transgene of interest, optionally a poly A sequence, and homology arms. In various embodiments, the nucleic acid comprises homology arm(s) (HA), a splice acceptor site, a promoter and a targeting site for a nuclease dependent cleavage system targeting molecule.

In various embodiments, the homology arms are between 35 and 1000 nucleotides. In various embodiments, the homology arms are from 50-900 nucleotides, 50-750 nucleotides, 100-600 nucleotides, 100-500 nucleotides, or 200-400 nucleotides. In various embodiments, the homology arms are 35, 40, 45, 50, 55, 60, 65, 70, 75, 80, 90, 100, 125, 150, 175, 200, 250, 300, 350, 400, 450, 500, 550, 600, 650, 700, 750, 800, 850, 900, 950 or 1000 nucleotides.

In various embodiments, the viral vector, plasmid, nanoplasmid or mini-circle comprises a nucleic acid further comprising a promoter next to or near the gene of interest/gene to be overexpressed. In various embodiments, the promoter is an endogenous T cell promoter. In various embodiments, the promoter is an MND promoter, a CMV promoter, a CAG promoter, a PGK promoter, a EF1A promoter, or a T cell-specific promoter.

### Cell Culture Methods

Mammalian T cells containing the recombinant protein-encoding DNA or RNA are cultured under conditions appropriate for growth of the cells and expression of the DNA or RNA. Those cells which express the recombinant protein can be identified, using known methods and methods described herein, and the recombinant protein can be isolated and purified, using known methods and methods also described herein, either with or without amplification of recombinant protein production. Identification can be carried out, for example, through screening genetically modified mammalian cells that display a phenotype indicative of the presence of DNA or RNA encoding the recombinant protein, such as PCR screening, screening by Southern blot analysis, or screening for the expression of the recombinant protein. Selection of cells which contain incorporated recombinant protein-encoding DNA may be accomplished by including a selectable marker in the DNA construct, with subsequent culturing of transfected or infected cells containing a selectable marker gene, under conditions appropriate for survival of only those cells that express the selectable marker gene. Further amplification of the introduced DNA construct can be effected by culturing genetically modified T cells under appropriate conditions (e.g., culturing genetically modified T cells containing an amplifiable marker gene in the presence of a concentration of a drug at which only cells containing multiple copies of the amplifiable marker gene can survive).

Genetically modified T cells expressing a recombinant protein of interest can be identified, as described herein, by detection of the expression product or cell surface markers.

Protein purification methods are known in the art and utilized herein for recovery of recombinant proteins from cell culture media. For example, methods of protein and antibody purification are known in the art and can be employed with production of the antibodies of the present disclosure. In some embodiments, methods for protein and antibody purification include filtration, affinity column chromatography, cation exchange chromatography, anion exchange chromatography, and concentration. The filtration step may comprise ultrafiltration, and optionally ultrafiltration and diafiltration. Filtration is preferably performed at least about 5-50 times, more preferably 10 to 30 times, and most preferably 14 to 27 times. Affinity column chromatography, may be performed using, for example, PROSEP® Affinity Chromatography (Millipore, Billerica, Mass.). In various embodiments, the affinity chromatography step comprises PROSEP®-vA column chromatography. Eluate may be washed in a solvent detergent. Cation exchange chromatography may include, for example, SP-Sepharose Cation Exchange Chromatography. Anion exchange chromatography may include, for example but not limited to, Q-Sepharose Fast Flow Anion Exchange. The anion exchange step is preferably non-binding, thereby allowing removal of contaminants including DNA and BSA. The antibody product is preferably nanofiltered, for example, using a Pall DV 20 Nanofilter. The antibody product may be concentrated, for example, using ultrafiltration and diafiltration. The method may further comprise a step of size exclusion chromatography to remove aggregates.

### Methods of Use

The engineered, gene edited T cell of the present disclosure is useful to as a cell-based therapy for protein deficiencies, enzymopathies, immunotherapy for infections, and autoimmune diseases.

In various embodiments, the engineered T cell(s) comprises an expression cassette comprising a polynucleotide to be overexpressed by the T cell. In various embodiments, the engineered T cell comprises an exogenous polynucleotide integrated within the genome.

In various embodiments, the engineered T cell comprises an expression cassette comprising a polynucleotide to be overexpressed by the T cell or an exogenous polynucleotide, wherein the polynucleotide encodes an enzyme, a cytokine, a chemokine, a T cell receptor, or a cell surface receptor. In various embodiments, the polynucleotide encodes an enzyme, a cancer antigen, a cytokine, a chemokine, a T cell receptor, or a cell surface receptor. In various embodiments, the polynucleotide encodes an antigen that is an enzyme, an autoimmune antigen, a microbial antigen, a viral antigen, or a bacterial antigen.

In various embodiments, the polynucleotide to be overexpressed encodes a therapeutic protein. In various embodiments the therapeutic protein is an enzyme, a cancer antigen, a cytokine, a chemokine, a T cell receptor, a cell surface receptor, a protein associated with a protein deficiency, or an extracellular membrane protein.

In various embodiments, the enzyme is an enzyme deficient in an enzymopathy. In various embodiments, the enzyme is L-lduronidase, lduronate-2-sulfatase, Heparan-N-sulfatase α-N-Acetylglucosaminidase AcetylCoA: N-acetyltransferase, N-Acetylglucosamine 6-sulfatase, Galactose 6-sulfatase, pGalactosidase, N-Acetylgalactosamine 4-sulfatase, β-Glucuronidase, hyaluronoglucosaminidase, Aspartylglucosaminidase, Acid lipase, Cystine transporter, Lamp, α-Galactosidase A. ceramidase, α-L-Fucosidase, Protective protein, β-glucosidase, Galactocerebrosidase, α-Glucosidase, β-Galactosidase, β-Hexosaminidase A, α-D-Mannosidase, β-D-Mannosidase, Arylsulfatase A, Neuraminidase, Saposin B, Phosphotransferase, Phosphotransferase γ-subunit, Palmitoyl protein thioesterase, Tripeptidyl peptidase 1, Acid sphingomyelinase, Cathepsin K, α-Galactosidase B, sialic acid transporter, Factor VII, or Factor VIII.

In various embodiments, the polynucleotide to be overexpressed encodes a collagen protein or a subunit thereof. In various embodiments, the polynucleotide is COL7A1 and the protein is collagen Type VII alpha 1 chain.

In various embodiments, the enzymopathy is selected from the group consisting of aspartylglucosaminuria, cholesterol ester storage disease, Wolman disease, metachromatic leukodystrophy, Danon disease, Fabry disease, Farber lipogranulomatosis, Farber disease, fucosidosis, galactosialidosis types I/II, Gaucher disease types I/II/III, globoid cell leukodystrophy, Krabbe disease, glycogen storage disease II, Pompe disease, GM1-gangliosidosis types I/II/III, GM2-gangliosidosis type I, Tay Sachs disease, GM2-gangliosidosis type II, Sandhoff disease, GM2-gangliosidosis, α-mannosidosis types I/II, β-mannosidosis, mucolipidosis type I, sialidosis types I/II, mucolipidosis types II/III, I-cell disease, mucolipidosis type IIIC, pseudo-Hurler polydystrophy, mucopolysaccharidosis type I, mucopolysaccharidosis type II, Hunter syndrome, mucopolysaccharidosis type IIIA, Sanfilippo syndrome type B, Sanfilippo syndrome type C, Sanfilippo syndrome type D, mucopolysaccharidosis type IIIB, mucopolysaccharidosis type IIIC, mucopolysaccharidosis type IIID, mucopolysaccharidosis type IVA, mucopolysaccharidosis type IVB, Morquio syndrome type A, Moriquio syndrome type B, mucopolysaccharidosis type VI, mucopolysaccharidosis type VII, Sly syndrome, mucopolysaccharidosis type IX, multiple sulfatase deficiency, neuronal ceroid lipofuscinosis, CLN1 Batten disease, CLN2 Batten disease, Niemann-Pick disease types A/B, Niemann-Pick disease, Niemann-Pick disease type C1, Niemann-Pick disease type C2, pycnodysostosis, Schindler disease types I/II, and sialic acid storage disease, hemophilia A, hemophilia B, Christmas disease, Factor VII deficiency, spinal muscular atrophy, and epidermolysis bullosa dystrophica.

In various embodiments, the enzymopathy is mucopolysaccharidosis type I (MPS I) and the gene of interest to be overexpressed is iduronidase. It is contemplated that the methods described herein ameliorate one or more symptoms of MPS I. In various embodiments, the one or more symptoms of MPS I are selected from the group consisting of reduction of glycosaminoglycan (GAG) species in tissues and/or urine, increase of IDUA expression in tissues, improved cognition, and reduced vacuolated endothelial cells/foam cells in tissue,

In various embodiments, the protein deficiency is epidermolysis bullosa dystrophica, also known as dystrophic epidermolysis bullosa, and the gene of interest to be overexpressed is collagen Type VII alpha 1 chain.

In various embodiments, the autoimmune antigen is associated with an autoimmune disease. In various embodiments, the autoimmune disease is selected form the group consisting of achalasia, Addison's disease, adult still's disease, agammaglobulinemia, alopecia areata, amyloidosis, ankylosing spondylitis, anti-gbm/anti-tbm nephritis, antiphospholipid syndrome autoimmune angioedema autoimmune dysautonomia autoimmune encephalitis autoimmune hepatitis, autoimmune inner ear disease (AIED), autoimmune myocarditis autoimmune oophoritis, autoimmune orchitis, autoimmune pancreatitis, autoimmune retinopathy autoimmune urticarial, axonal & neuronal neuropathy (AMAN), Baló disease, Behcet's disease, benign mucosal pemphigoid (Mucous membrane pemphigoid), bullous pemphigoid, Castleman disease (CD), Celiac disease, Chagas disease, chronic inflammatory demyelinating polyneuropathy (CIDP), chronic recurrent multifocal osteomyelitis (CRMO), Churg-Strauss syndrome (CSS) or eosinophilic granulomatosis (EGPA), cicatricial pemphigoid, Cogan's syndrome, cold agglutinin disease, complex regional pain syndrome (formerly known as reflex sympathetic dystrophy), congenital heart block, coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, granulomatosis with polyangiitis, Graves' disease, Guillain-Barre syndrome, Hashimoto's thyroiditis, hemolytic anemia, Henoch-Schonlein purpura (HSP), herpes gestationis or pemphigoid gestationis (PG), hidradenitis suppurativa (HS) (acne inversa), IgA nephropathy, IgG4-related sclerosing disease, immune thrombocytopenic purpura (ITP), inclusion body myositis (IBM), interstitial cystitis (IC), juvenile arthritis, juvenile diabetes (Type 1 diabetes), juvenile myositis (JM), Kawasaki disease, Lambert-Eaton syndrome, lichen planus, lichen sclerosus, ligneous conjunctivitis, linear IgA disease (LAD), lupus, Lyme disease chronic, Meniere's disease, microscopic polyangiitis (MPA), mixed connective tissue disease (MCTD), Mucha-Habermann disease, multifocal motor neuropathy (MMN) or MMNCB, multiple sclerosis, myasthenia gravis, myelin oligodendrocyte glycoprotein antibody disorder, myositis, narcolepsy, neonatal lupus, neuromyelitis optica/devic disease, neutropenia, ocular cicatricial pemphigoid, optic neuritis, palindromic rheumatism (PR), PANDAS (Pediatric autoimmune neuropsychiatric disorders associated with streptococcus infections), paraneoplastic cerebellar degeneration (PCD), paroxysmal nocturnal hemoglobinuria (PNH), pars planitis (peripheral uveitis), Parsonage-Turner syndrome, pemphigus, peripheral neuropathy, perivenous encephalomyelitis, pernicious anemia (PA), POEMS syndrome, polyarteritis nodosa, polyglandular syndromes type I, II, III, polymyalgia rheumatic, polymyositis, postmyocardial infarction syndrome, postpericardiotomy syndrome, primary biliary cholangitis, primary sclerosing cholangitis, progesterone dermatitis, progressive hemifacial atrophy (PHA), Parry romberg syndrome, psoriasis, psoriatic arthritis, pure red cell aplasia (PRCA), pyoderma gangrenosum, Raynaud's phenomenon, reactive arthritis, relapsing polychondritis, restless legs syndrome (RLS), retroperitoneal fibrosis, rheumatic fever, rheumatoid arthritis, sarcoidosis, Schmidt syndrome or autoimmune polyendocrine syndrome type II, scleritis, scleroderma, Sjögren's Disease, stiff person syndrome (SPS), Susac's syndrome, sympathetic ophthalmia (SO), Takayasu's arteritis, temporal arteritis/giant cell arteritis, thrombocytopenic purpura (TTP), thrombotic thrombocytopenic purpura (Ttp), thyroid eye disease (Ted), Tolosa-Hunt syndrome (THS), transverse myelitis, type 1 diabetes, ulcerative colitis (UC), undifferentiated connective tissue disease (UCTD), uveitis, vasculitis, vitiligo, Vogt-Koyanagi-Harada disease, and warm autoimmune hemolytic anemia.

In various embodiments, an engineered T cell includes an expression cassette comprising a polynucleotide encoding a cytokine. The cytokine includes, but is not limited to, IL-2, IL-4, IL-7, IL-12, IL-13, IL-15, IL-17, IL-21, IL-32, IL-33, IFN-γ, or combinations thereof.

In various embodiments, the method further comprises inactivating a gene encoding the target antigen of interest in the non-human animal. In various embodiments, the gene encoding the target antigen is inactivated using a nuclease-dependent cleavage system.

### Kits

The polynucleotides, plasmid system or vectors described herein may be provided in a kit. The kits may include, in addition to the polynucleotide, plasmid system or vector, any reagent which may be employed in the use of the system. In one embodiment, the kit includes reagents necessary for transformation of the vectors into mammalian cells. The kit may include growth media or reagents required for making growth media, for example, DMEM for growth of mammalian cells. Components supplied in the kit may be provided in appropriate vials or containers (e.g., plastic or glass vials). The kit can include appropriate label directions for storage, and appropriate instructions for usage.

### EXAMPLES

### Example 1—Materials and Methods

Culture media: OPTIMIZERS™ T-Cell Expansion Basal Medium (Gibco), 2.6% OpTmizer™ T-Cell Expansion Supplement (Gibco), 2.5% CTS™ Immune Cell Serum Replacement (Gibco)

Recovery media: 400 mLs OPTIMIZER™ T-Cell Expansion Basal Medium (Gibco), 10.4 mLs 2.5% CTS™ Immune Cell Serum Replacement (Gibco), 10 mLs Immune Cell SR, 4 mLs 1% L-Glutamine (Gibco), 7.3 mL of 10 mM 10 mM N-Acetyl-L-cysteine (Sigma).

1×TCM (Complete T cell media): 400 mLs OPTIMIZER™ T-Cell Expansion Basal Medium (Gibco), 10.4 mLs 2.5% CTS™ Immune Cell Serum Replacement (Gibco), 10 mLs Immune Cell SR, 4 mLs 1% L-glutamine (Gibco), 4 mLs 1% penicillin/streptomycin (Millipore), 7.3 mL of 10 mM 10 mM N-Acetyl-L-cysteine (NAC) (Sigma), 20 uL each of 300 IU/ml recombinant human IL-2 (Peprotech), 5 ng/ml recombinant human IL-7 (Peprotech) and 5 ng/ml recombinant human IL-15 (Peprotech).

Stimulation of T cells for engineering: Initially, 1×TCM is pre-equilibrated at 1 mL per 1×10⁶ cells in a tissue culture plate (G-Rex 24-well cell culture plate (Wilson Wolf) at least 15 minutes before use. 2 mL of 1×TCM is aliquoted into a 15 ml conical tube, incubated in 37° C. water bath for 10 minutes. Cryopreserved T cells are thawed and once thawed, 1 mL of pre-warmed media from the 15 mL conical tube s dripped into the cryopreserve tube containing T cells. The whole volume of cell suspension is transferred from the cryopreserve tube back to the 15 mL conical tube with pre-warmed media and the volume brought to 15 mL with 1×PBS. Cells are centrifuged at 400×g for 5 minutes. While waiting, DYNABEADS™ Human T Expander (Fisher Scientific, Hampton, NH) are vortexed before taking 50 ul of the beads per 1×10⁶ T cells to obtain 2:1 ratio (beads:cells). The beads are washed once on a DYNAMAG™-2 magnet (Fisher Scientific, Hampton, NH) with 1×PBS. 100 ul of 1×TCM are used to resuspend the beads and the beads transferred into the tissue culture plate containing pre-equilibrated 1×TCM. After centrifugation of the cells, supernatant is removed without disturbing the pellet and the pellet resuspended with a complete media to a concentration of 1×10⁶ cell/mL. The cell suspension is transferred into the tissue culture plate containing 1×TCM and the beads and placed in the tissue culture incubator at 37° C., 5% CO₂ with humidity for 36 hours (plasmid engineering) or 48 hours (viral engineering).

T cell engineering: T cells are stripped from DYNABEADS™ by taking 1 mL of the cells from the tissue culture plate to a well of a 24-well plate, the pipet tips containing the cells are pressed at 45 degrees angle to the bottom of the plate, pipette up and down 10 times, transfer the stripped cells into a clean 15 mL conical. Transfer the 15 mL conical to an EASYEIGHTS™ EASYSEP™ Magnet (STEMCELL Technologies, Vancouver, Canada), incubate at room temperature for 1 minute without cap. This allows the DYNABEADS™ to stick to the wall of the tube. The cell suspension is transferred into a tissue culture plate and placed in the tissue culture incubator until use. 300 ul of recovery media per 1×10⁶ cells is transferred into a well of a tissue culture plate and 700 ul of 1×TCM per 1×10⁶ cells is transferred into another well of the tissue culture plate. Media is pre-equilibrated at least 15 minutes before use.

2 μL of the CRISPR/Cas9 substrate or PBS is transferred to appropriate PCR tubes. 1×10⁶ T cells are transferred (per electroporation reaction) into a 15 mL conical tube and centrifuged at 200×g for 10 minutes. The supernatant is removed and cell pellet resuspended with 14 mL 1×PBS. The tube is centrifuged at 200×g for 10 minutes, and supernatant removed completely without disturbing the cell pellet. Cell pellet is resuspended with 20 uL nucleofection reagent per 1×10⁶ T cells. The 20 ul cell suspension is transferred to the PCR tubes containing 2 μL of CRISPR/Cas9 substrate to make electroporation reaction, do not mix. The whole volume of the electroporation reaction is transferred from the PCR tube to a cuvette of a 16-cuvette strip, do not mix. The cells are electroporated using EO-115 (for viral approach) or (FI-115 for plasmid approach) on a Lonza 4D system, and then rested in the cuvette at room temperature for 15 minutes. 80 ul of the pre-equilibrate recovery media is transferred into the electroporated cells and the cells immediately transferred to a well containing 300 ul recovery media. The plate is placed back into the tissue culture incubator and incubated for 30 minutes.

While waiting, 12.5 ul of DYNABEADS per 1×10⁶ electroporated cells is washed and transferred into the wells containing 700 μL of 1×TCM. After 30 minutes, 700 μL of 1×TCM with DYNABEADS™ is transferred into the well containing cells in the recovery media. The final concentration of the cells at this step is 1×10⁶ cell/ml. The plate is placed back to the tissue culture incubator for 24-48 hours. If using plasmid as a donor template, add DNase 1 at a final concentration of 1 μg/mL media. Immediately transfer rAAV viral vector to the viral approach conditions at 500,000 multiples of infection (MOI).

At least 1-5 million engineered cells are transferred from the tissue culture plate to a well of a 24-well G-Rex containing 6.6 ml pre-equilibrated 1×TCM. Half of the media in the G-Rex well is replaced without disturbing the cells every 3 days or when the media turn orange/yellow-ish. Cells are counted on day 6, and 200,000 cells taken for flow cytometry analysis and sampling the culture media for IDUA assay.

The remaining cells can be used for downstream analysis and/or for engraftment. For engraftment, cells are harvested and strip the dynabeads stripped as described previously. Cells are centrifuged at 200×g for 10 minutes, supernatant removed and cells resuspended with 1×PBS for injection.

CRISPR/Cas9: Chemically modified sgRNAs 1. AAVS1 sgRNA sequence: GTCACCAATCCTGTCCCTAG (SEQ ID NO: 1)—targets AAVS1 locus of T cells. 2. CLEANCAP™ S. pyogenes (S.p.) Cas9 mRNA. 2. AAVS1 sgRNA sequence: GTCACCAATCCTGTCCCTAG (SEQ ID NO: 1)—targets AAVS1 locus of B cells; 3. Universal sgRNA sequence: GGGAGGCGTTCGGGCCACAG (SEQ ID NO: 2)—targets Universal target sequence on a nanoplasmid for HMEJ mechanism; 3. CLEANCAP™ S.p. Cas9 mRNA.

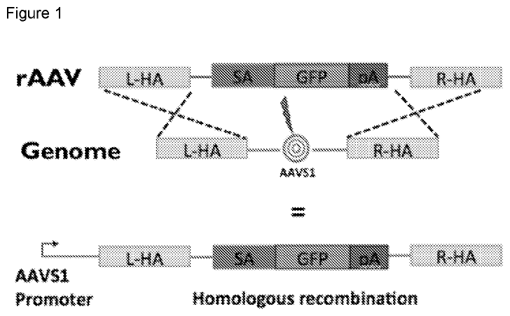

rAAV vector constructs: Targets AAVS1 locus of T cells 1. rAAV AAVS1 SA-EGFP (FIG. 1). 2. rAAV AAVS1 MND-IDUA-RQR8 (FIG. 4A).

Plasmid construct constructs: Target AAVS1 locus of T cells, pAAV AAVS1 MND-IDUA-RQR8 (FIG. 10).

Electroporation: Lonza 4D electroporator system; AMAXA™ P3 Primary Cell 40-NUCLEOFECTOR™ X kit contains: 2×16-cuvette strips, P3 primary Cell Solution, Supplement 1, DNase 1 solution 1 mg/ml (STEMCELL Technologies).

Flow cytometry: Cell were stained with PerCP-conjugated CD4, viability dye (eF780), Cy7PE-conjugate anti-CD45RO and PE-conjugated anti-CD34 (anti-RQR8) antibody clone QBEND/10.

### Example 2

### Engineering T Cells to Express a Therapeutic Protein

A successful T cell engineering process inserts a transgene on the T cell genome and allows T cells to overexpress a transgene of interest. CRISPR/Cas9 mediated T cell engineering field is primarily focusing on expressing chimeric antigen receptor (CAR) for cancers therapies (Li, et al. (2020). Brief. Funct. Genomics 19, 175-182; Rupp et al. (2017) Sci. Rep. 7, 737; Choi et al. (2019) J. Immunother. Cancer 7, 1-8) autoimmune diseases and infections (Maldini et al. (2018). Nat. Rev. Immunol. 2018 1810 18, 605-616.). Experiments engineering T cells to express or overexpress proteins or enzymes and use of the engineered T cells as a therapy for protein deficiencies or enzymopathies, e.g., mucopolysaccharidosis type I (MPS I), has not been described.

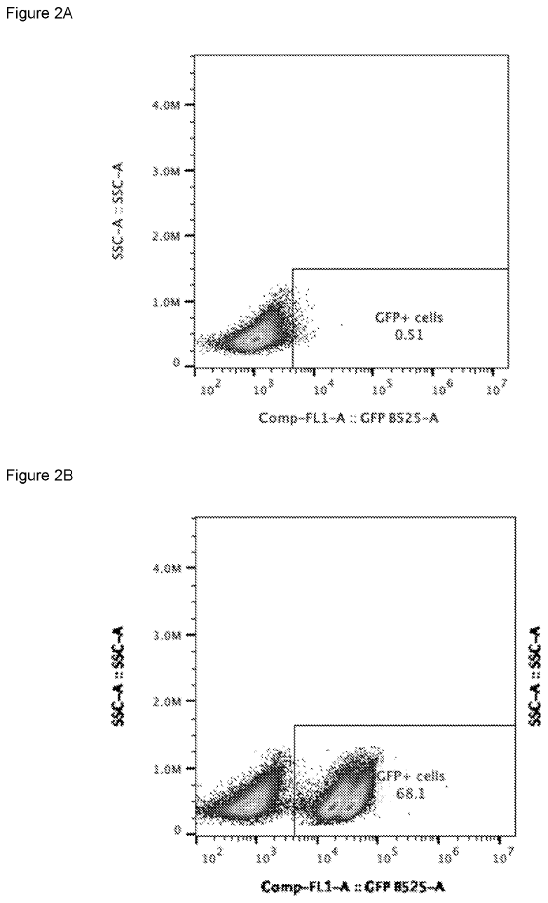

To investigate CRISPR/Cas9 together with recombinant adeno-associated viral (rAAV) vector donor template to mediate site-specific insertion of a transgene, a promoterless GFP expressing cassette was constructed with homology arms (Has) targeting the AAVS1 locus and packaged it into a rAAV vector (rAAV AAVS1 SA-GFP; FIG. 1). T cells were stimulated for 48 hours before engineering. Upon 48-hour stimulation, CRISPR/Cas9 reagents were transfected in the cells via an electroporator. The electroporated cells were immediately transduced with rAAV AAVS1-SA-GFP at 500,000 MOIs. The successful integration of GFP cassette into the T cell genome allows GFP to be expressed under the regulation of the endogenous promoter of the AAVS1 locus (FIG. 1), while non-integrating DNA template will not express GFP. GFP-positive T cells were not observed in the vector-only sample (FIG. 2A) and up to 68% GFP-positive T cells were observed post engineering (FIG. 2B). This indicates that CRISPR/Cas9 and rAAV donor template can efficiently mediated transgene integration and expression in T cells.

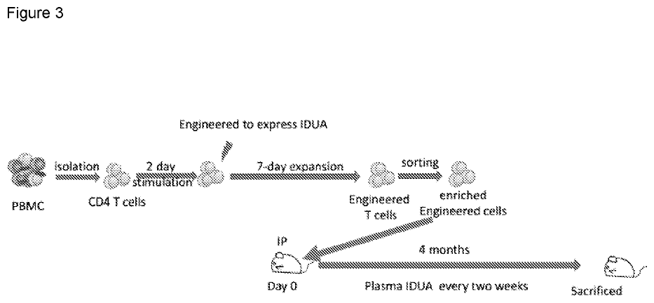

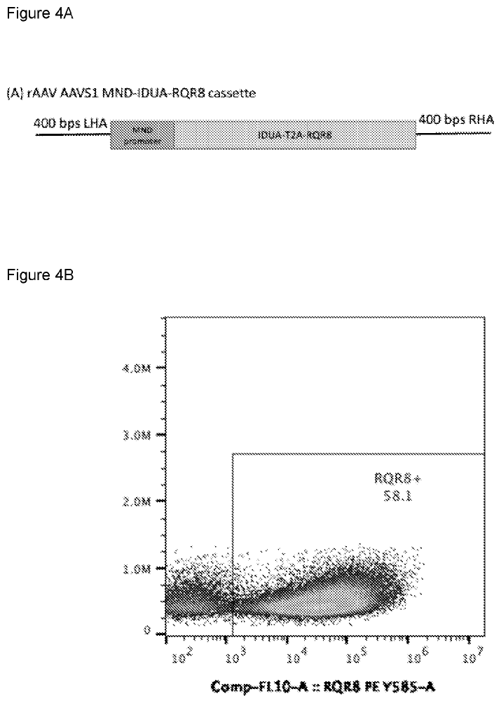

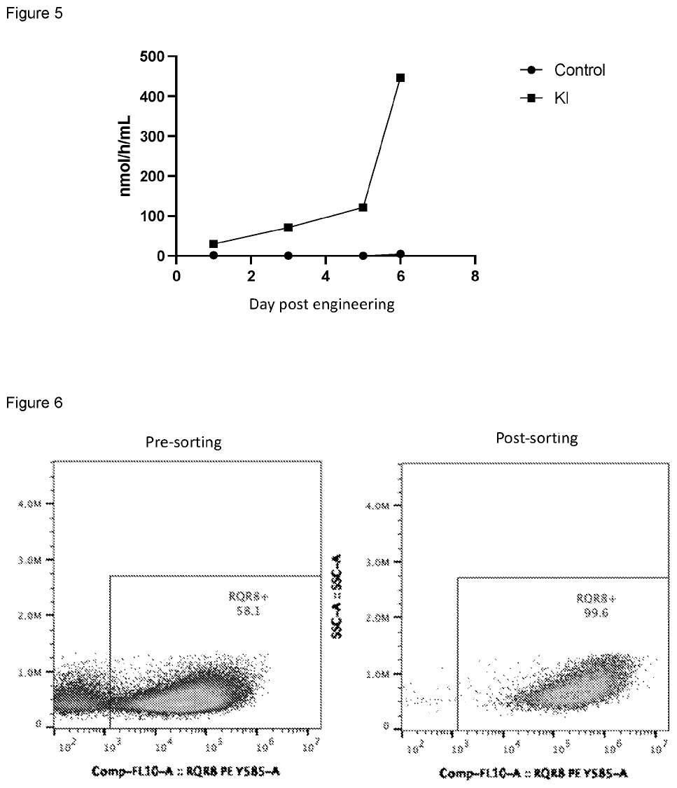

A pilot study to engraft engineered T cells in a mouse model of MPS I (FIG. 3) was conducted. First, it was tested whether T cells can be engineered to express IDUA by using an existing rAAV AAVS1 MND-IDUA-RQR8 vector (FIG. 4A) as a donor template to engineer T cells. Up to 58% RQR8-positive T cells were observed (FIG. 4B), indicating efficient engineering efficiency. In addition, media containing engineered T cells showed elevated IDUA activity levels (˜400 nmol/hr/ml), while only background IDUA levels were observed in media from the control non-engineered T cells (FIG. 5). This indicates that the RQR8-positive T cells are expressing and secreting IDUA in the culture media. Next, T cells were enriched using RQR8-positive sorting strategy, yielding 99.8% purity (FIG. 6).

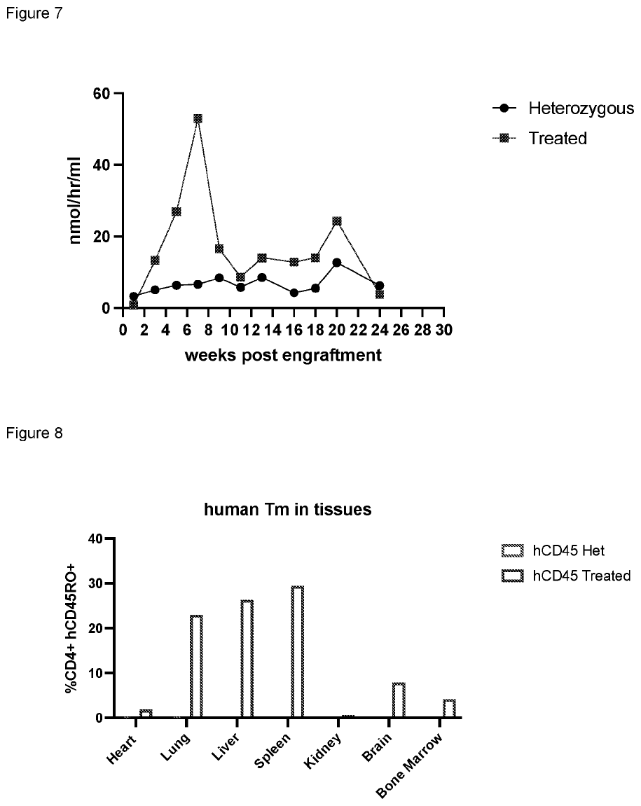

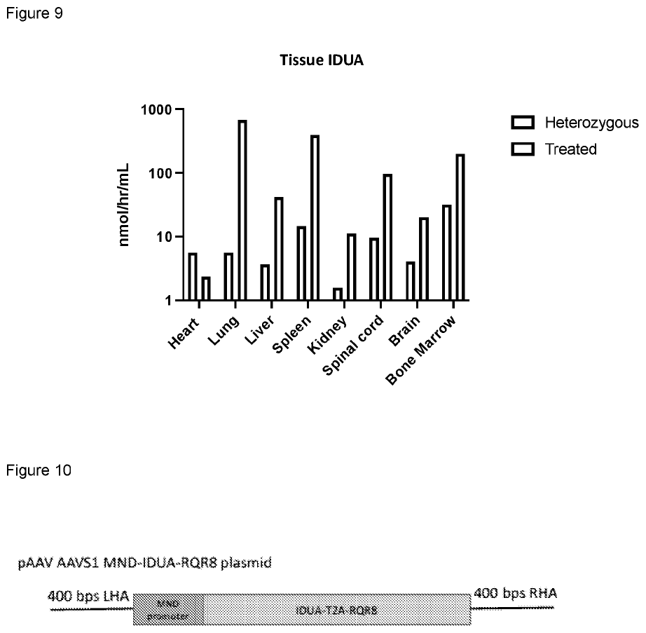

8×10⁶ engineered T cells were engrafted into a 3-week-old NOD-SCID-IDUA deficient mouse (NSG-IDUA−/−; NSG-MPSI). MPS I is an autosomal recessive disease. In a mouse model, the affected mice must carry two IDUA knocked out alleles while the heterozygous is considered a “wild-type”. Plasma IDUA was monitored in the treated NSG-MPSI mouse to a heterozygous littermate for a course of 24 weeks. Blood was collected in the EDTA tubes and processed for plasma at the indicated time points (FIG. 7) to measure IDUA activity in the plasma. A slight elevation of IDUA was observed in the plasma as early as week 1 post engineering (FIG. 7), continued to increase and peaked at week 6 post engraftment (FIG. 7). IDUA started to drop off to the level similar to that of the heterozygous littermate and persisted throughout the study period (at least 24 weeks) post engraftment (FIG. 7). At the end of the study (week 25), the mice were sacrificed, the vital organs were collected and processed to determine the presence of T cells as well as IDUA levels in these tissues. Flow cytometry showed human T cells in all tested organs (FIG. 8). A high ratio of human T cells to mouse lymphocytes were observed in the lung, the liver, and the spleen (FIG. 8). This is likely due to the presence of secondary lymph nodes found in these organs. This indicates that engineered T cells can persist at least 24 weeks in the treated animal. No human T cells were observed in the heterozygous littermate. T cell infiltration in the brain and the bone marrow was also observed, suggesting that T cells migrate to the brain and the bone marrow. Tissue IDUA showed supraphysiological levels of IDUA in all tested organs except the heart (50% of heterozygous level; FIG. 9). This indicates that engineered T cells expressed and secrete IDUA for systemic cross collection in the treated animal.

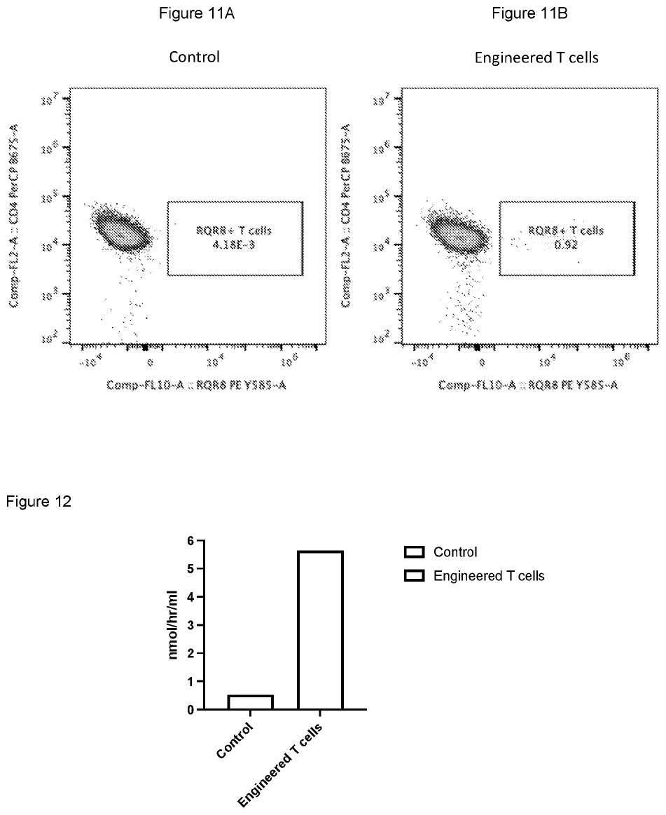

The ability to use plasmid to non-virally engineer T cells was also tested. CRISPR/Cas9 together with plasmid DNA donor template (FIG. 10) were transfected into T cells via electroporation. The cells were cultured and expanded for 5 additional days in culture. Low engineering efficiency of T cells using the plasmid was observed (FIG. 11). The lower engineering efficiency is due to pAAV as not an optimal plasmid for engineering. Nonetheless, IDUA activity in the media culture of engineered T cells showed elevated IDUA activity when compared to the media culture of the control T cells (FIG. 12). This indicates that plasmid can be used as a donor template for CRISPR/Cas9 mediated targeted insertion and expression of the transgene.

These results show engineering protocols for CRISPR/Cas9 mediated targeted insertion of transgenes in T cells, demonstrating that engineered T cells can express and secrete proteins/enzymes inserted into the genome. Engineered cells can be transplanted and persist in an animal model for at least 24 weeks. In addition, IDUA-expressing T cells can produce enzyme to a level that is comparable to the non-diseased heterologous NSG littermate. Moreover, IDUA enzyme can be taken up by tissues in the tested vital organs of the NSG-MPSI mouse, resulting in supra-physiological levels of the enzyme. Finally, it was shown that T cells can migrate to the brain of the MPS I animal, suggesting potential use of engineered T cells for a disease with neurologic involvement.

### Example 3—In Vivo Analysis of Therapeutic Gene Expression

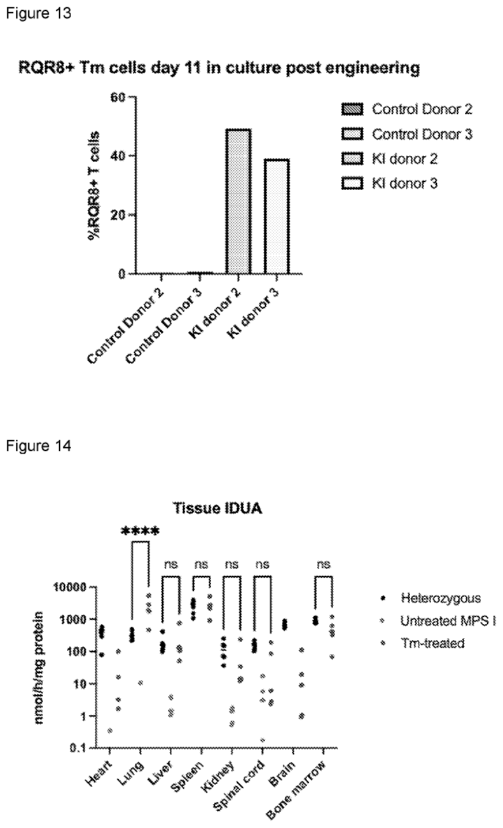

To investigate CRISPR/Cas9 together with recombinant adeno-associated viral (rAAV) vector donor template to mediate site-specific insertion of a therapeutic transgene, CRISPR-Cas9 combined with rAAV6 was used for site-specific insertion of a SA-STOP-pA-uMND-IDUA-RQR8 cassette. T cells were stimulated for 48 hours before engineering. Upon 48-hour stimulation, CRISPR/Cas9 reagents were transfected in the cells via an electroporator. The electroporated cells were immediately transduced with rAAV SA-STOP-pA-uMND-IDUA-RQR8 at 500,000 MOIs as described above. The DNA donor template, delivered by rAAV6, targeted the AAVS1 locus in memory T cells. Up to 49% RQR8+ at day 11 post engineering, while no detectible RQR8+ cells were detected in the non-engineered control (FIG. 13). This indicates that the CRISPR-Cas9 combined with the rAAV6 can mediate efficient insertion and expression of IDUA-RQR8 within the AAVS1 locus in T cells.

In order to determine the ability of engineered T cells to express therapeutic protein in vivo Engineered Tm cells from donor two above (pAAV AAVS1 MND-IDUA-RQR8) were engrafted into 8 NSG-IDUA deficient mice. Eight NSG-IDUA deficient mice were injected with the engineered human Tm cells at 1e\hat{ }6 cells per mouse. Increased levels of IDUA activity were observed in all the tested vital organs 12 weeks post engraftments (FIG. 14). There was no significant difference of IDUA levels of the heterozygous NSG mice and the Tm treated NSG-IDAU deficient mice in liver, spleen, kidney, spinal cord, and bone marrow. A higher level of IDUA activity was measured in the lung tissue within the Tm treated NSG-IDUA deficient mice compared to heterozygous NSG mice. Overall, higher levels of IDUA were observed in the Tm treated NSG-IDUA deficient mice than the untreated NSG-IDUA deficient mice. This indicates the measured IDUA in the Tm treated NSG-IDUA deficient mice is secreted from the engineered human Tm cells.

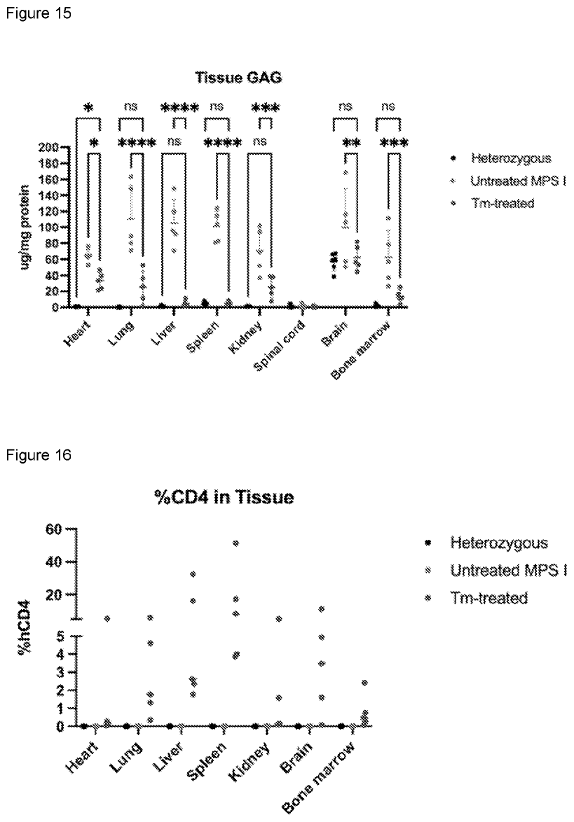

Twelve weeks post injections, tissue GAG contents were significantly lower in the heart, lung, liver, spleen, kidney, brain, and bone marrow. The spinal cord did not have a significant difference in GAG contents between the heterozygous, NSG-IDUA deficient, and treated NSG-IDUA deficient mice (FIG. 15). Treated NSG-IDUA deficient mice had no significant difference in GAG content levels from heterozygous NSG mice in the Lung, liver, spleen, kidney, brain, and bone marrow. This indicates the secreted IDUA from the engineered Tm cells are decreasing the GAG contents in vital organs.

Human CD4 positive cells were observed in all the tested vital organs of the Tm treated NSG-IDUA deficient mice at 12 weeks post injections (FIG. 16). No CD4+ cells were observed in the untreated NSG-IDUA deficient or heterozygous mice. This indicates these cells are able to persist in pathological IDUA deficient tissues for at least twelve weeks.

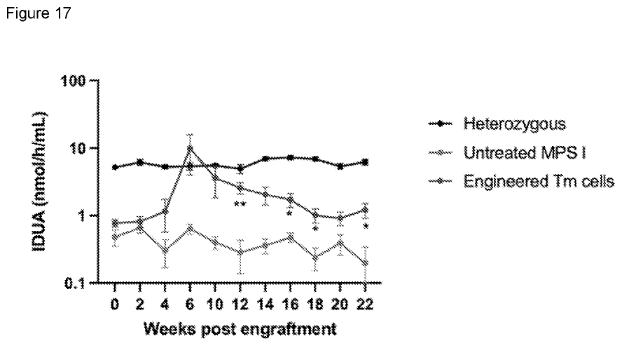

IDUA levels were also analyzed. Measurable IDUA activity was observed through all observed time points in the plasma of tm treated NSG-IDUA deficient mice (FIG. 17). Six weeks after injections IDUA activity levels peaked in the treated mice surpassing heterozygous IDUA activity levels. Throughout 22 weeks, IDUA levels in plasma was significantly higher compared to the untreated NSG-IDUA deficient mice.

Pathological urine GAG content in treated NSG-IDUA deficient mice was also determined. NSG-IDUA deficient mice were injected with the engineered human Tm cells through at 1e\hat{ }6 cells per mouse. At 6, 12, and 18 weeks post injection, urine GAG contents were significantly lower (FIG. 18). Week 6 and 12, had no significant difference of urine GAG contents in the treated NSG-IDUA deficient mice and heterozygous mice. This indicates the secreted IDUA from the engineered Tm cells are decreasing the GAG contents throughout the NSG-IDUA deficient mice.

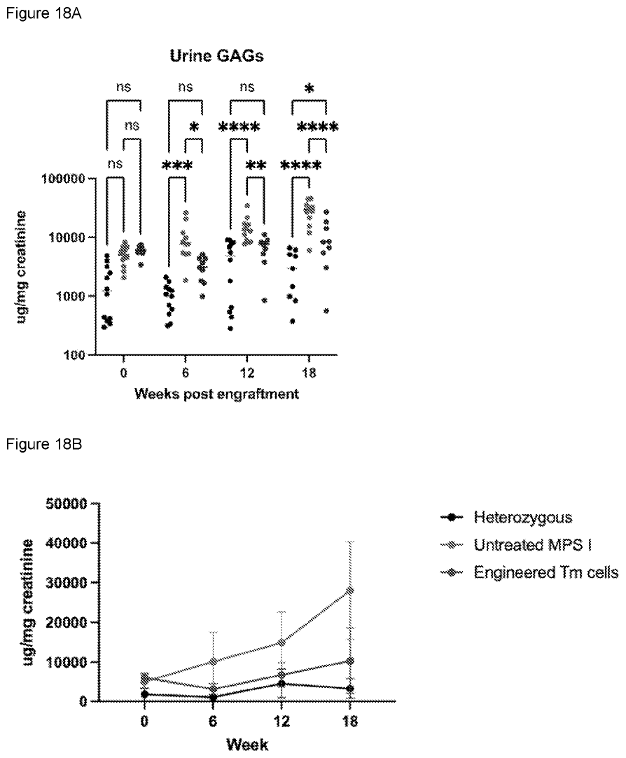

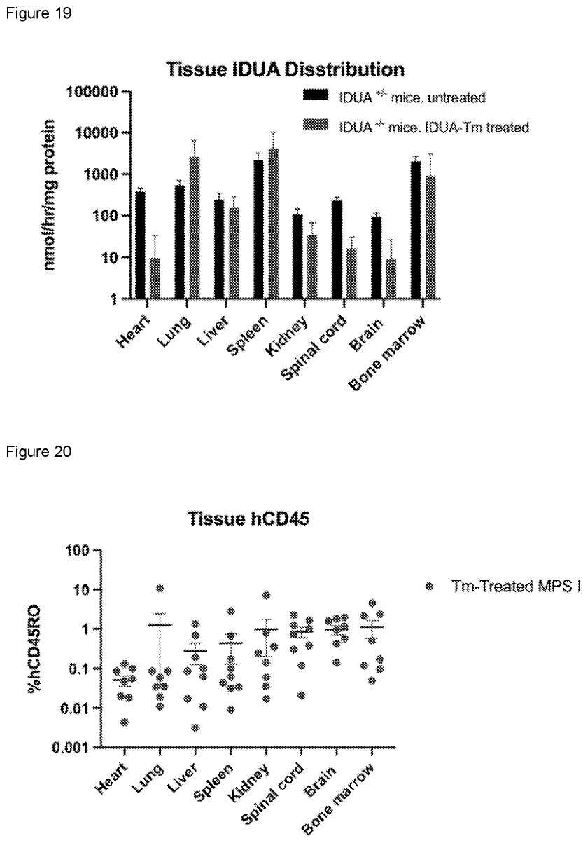

Analysis of tissue IDUA showed that increased levels of IDUA activity were observed in all the tested vital organs 22 weeks post engraftments (FIG. 19). IDUA activity in the treated NSG-IDUA deficient mice surpassed the heterozygous IDUA activity levels in the lung and spleen. Overall, higher levels of IDUA were observed in the Tm treated NSG-IDUA deficient mice than the untreated NSG-IDUA deficient mice. This indicates the measured IDUA in the Tm treated NSG-IDUA deficient mice is secreted from the engineered human Tm cells for a minimum of 22 weeks.

Human CD45 positive cells were overserved in all the tested vital organs of the Tm treated NSG-IDUA deficient mice at 22 weeks post injections (FIG. 20). No CD45 positive cells were observed in the untreated NSG-IDUA deficient or heterozygous mice. This indicates these cells are able to persist in pathological IDUA deficient tissues for at least 22 weeks.

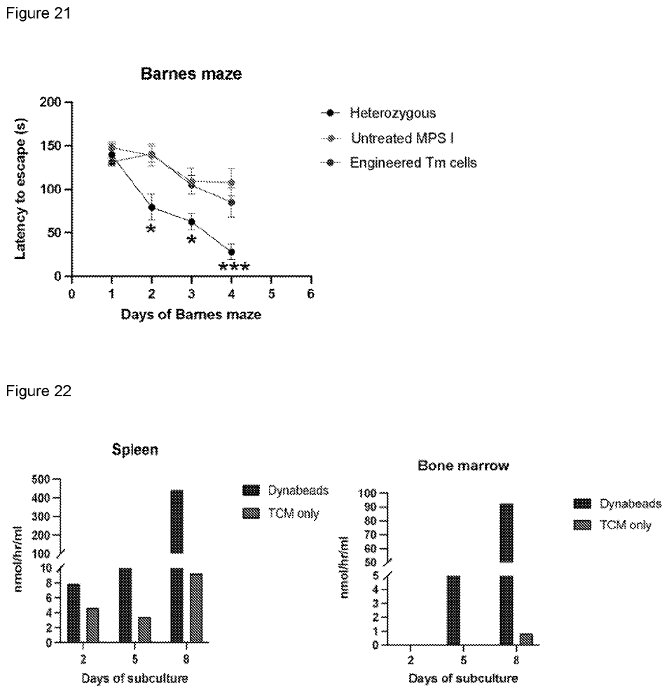

22 Weeks post cell injections, mice underwent a Barnes Maze neurocognitive assay. Untreated NSG-IDUA deficient mice had the longest latency to escape the Barnes Maze (FIG. 21). Treated NSG-IDUA mice had a decreased trend of time to find escape hole over the course of 4 testing days. Heterozygous mice performed the best at testing days 2, 3 and 4. This indicates there may be a beneficial neurocognition effect from the engineered human Tm cells.

Engrafted human cells were isolated 22 weeks post engraftment in NSG-IDUA deficient mice. These cells were cultured in activating T cell media for eight days. Cell expansion was observed in T cell media (TCM) and with Dynabead stimulation. Dynabead stimulation produced the highest IDUA activity secreted into culture media (FIG. 22). This indicates these cells have not become senescent or exhausted. This also indicates the engineered cells have to potential to be re-stimulated.

### Histological Profiles

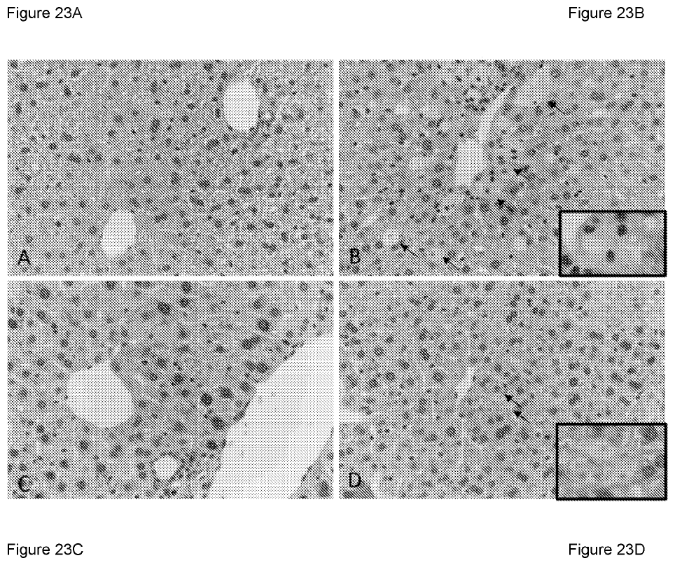

Liver tissue in treated and untreated animals was stained by H&E staining after 22 weeks. The NSG-IDUA deficient mice have individual and small clusters of round cells with amphophilic, finely vacuolated cytoplasm (indicative of lysosomal storage pathology), often with peripherally located nucleus (interpreted as foam cells) (FIG. 23). These foam cells were predominantly clustered around the central veins, and less commonly around portal triads and randomly throughout the liver. NSG-IDUA deficient mouse with engineered Tm cells (FIG. 23C) have no pathological vacuolation comparable to the Heterozygous NSG mice (FIG. 23A). FIG. 23D has an attenuation of the pathological intracytoplasmic vacuolation of the NSG-IDUA deficient mouse (FIG. 23B) with scattered low number of individual foam cells.

Staining of brain tissue after 22 weeks showed a NSG-IDUA deficient mouse (FIG. 24B) has rare neurons with finely vacuolated cytoplasm predominantly within the thalamus. This is indicative of lysosomal storage pathology. There are rare small clusters of foam cells around small-caliber blood vessels within the cerebrum and leptomeninges, and rare endothelial cells have a finely vacuolated cytoplasm. Treated NSG-IDUA deficient mouse with engineered Tm cells (FIG. 24C) appear to have a fewer number of vacuolated endothelial cells/foam cells.

At the cellular level, lysosomal storage disorders manifest in greater size and numbers of lysosomes. We expect afflicted tissue to feature frequent clusters of finely vacuolated macrophages, termed foam cells, in addition to increased vacuolation of other cell types. IDUA immunohistochemistry (IHC) stain of liver (FIG. 25) demonstrates that (A) Untreated MPS I afflicted tissue with foam cell clusters indicated by black boxes (FIG. 25A) while in (B) Treated MPS I afflicted tissue. Black arrows show round lymphocytes with moderate IDUA immunopositivity are observed (black arrows, FIG. 25B) and also exhibit irregularly shaped Kupffer cells (macrophages) with strong IDUA immunopositivity (red arrows, inset displays Kupffer cell). Any round lymphocytes in treated mice are interpreted as therapeutic cells due to the NSG background of the disease model. Through IDUA IHC, it was observed that IDUA from the treatment is localized to a few cell types and rarely seen in hepatocytes. Despite this, foam cells are almost entirely eliminated from all treated mice, indicating amelioration of the disease phenotype.

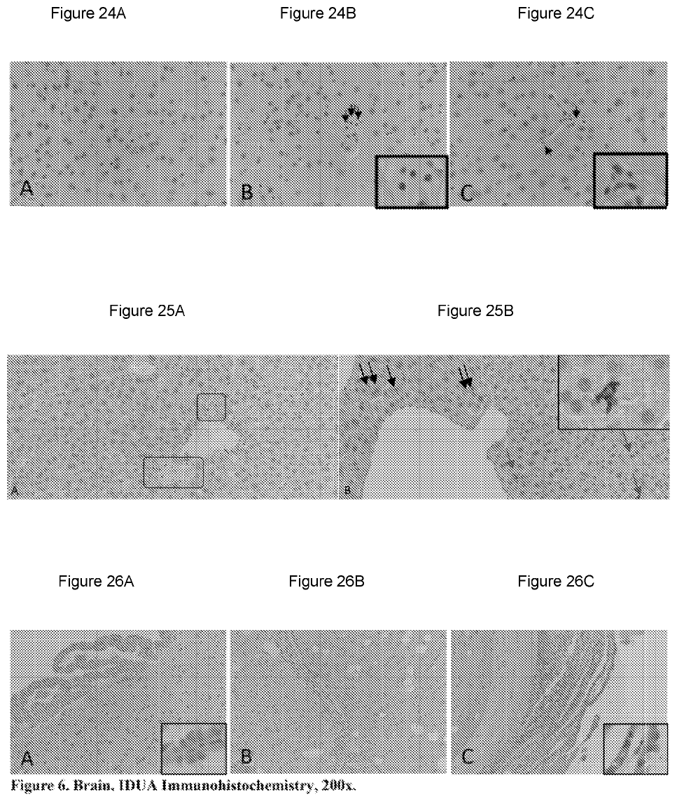

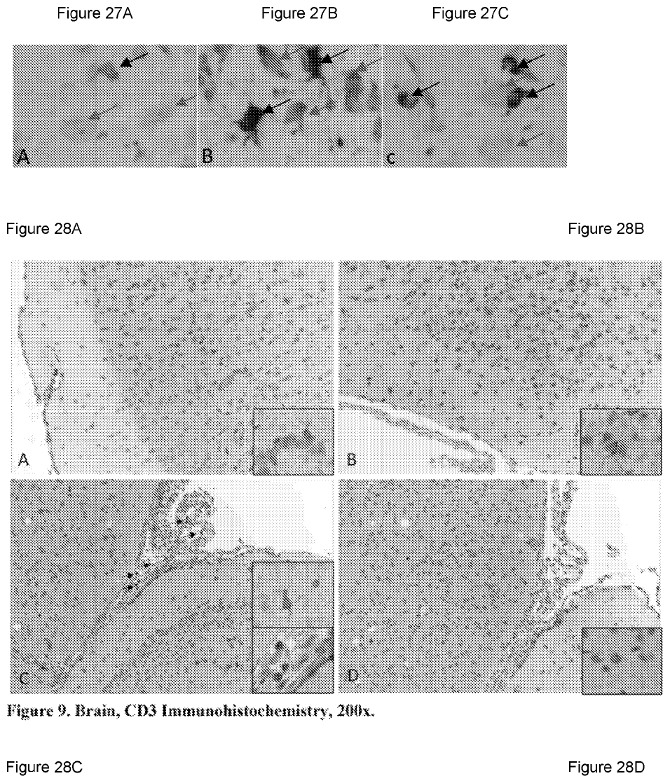

Lysosomal membrane associated protein 1 (LAMP-1) IHC in the liver demonstrates an ameliorated disease phenotype (FIG. 26). While endothelial cells and bile duct epithelium in the treated mice retain the intense, globular staining seen in untreated afflicted tissue, LAMP-1 staining in hepatocytes and sinusoids is coarse and granular like in healthy tissue. The blood brain barrier prevents a significant challenge to existing MPS I therapies, yet the present engineered Tm approach led to a healthier brain shown in FIG. 27. While astrocytes in this treated mouse are intensely immunopositive as in the untreated mouse, its neurons and surrounding tissue have greatly reduced immunopositivity. This implies fewer and smaller lysosomes, and an attenuated histopathology.

Further analysis of brain tissue from treated and untreated mice (FIG. 28) shows that NSG-IDUA deficient mice (FIG. 28C) with engineered Tm cells are exhibiting greater quantities of hIDUA immunopositivity. This hIDUA immunopositivity was more prevalent in the choroid plexus of the treated NSG-IDUA deficient mice than the heterozygous and NSG-IDUA untreated mice. hIDUA immunopositivity of the heterozygous and NSG-IDUA deficient mice indicate background hIDUA staining.

A summary of the histological results in shown in Table 1 below.

TABLE 1
Summarized hIDUA Immunohistochemistry Results*

| Mouse | | |
|---|---|---|
| Phenotype | Liver | Brain |
| Het | 1/4 | 3/4 |
| | (rare | (rare choroid |
| | hepatocytes) | plexus cells) |
| MPS Untreated | 0/4 | 1/4 ** |
| | | (rare choroid |
| | | plexus cells) |
| MPS Treated | 4/4 (few endothelial cells, | 2/4 |
| | Kupfer cells, and rare | (few choroid |
| | hepatocytes) | plexus cells) |

*Values represent the number of animals in which any positive staining was detected over the total number of animals in the group.
** The choroid plexus was not present in 2 of 4 of the mice in this group.

Assessment of the level of CD3+ T cells was also carried out. Heterozygous and NSG-IDUA deficient mice (FIG. 28A-B) depict the nonspecific CD3+ immunopositivity background. Treated NSG-IDUA deficient mice with engineered Tm cells (FIG. 28C) contain CD3+ lymphocytes indicated by black arrows. These CD3+ cells are localized within the leptomeninges of the brain tissue.

TABLE 2
Summarized CD3 epsilon Immunohistochemistry Results*

| Mouse | | |
|---|---|---|
| Phenotype | Liver | Brain |
| Het | 0/4 | 0/4 |
| MPS Untreated | 0/4 | 0/4 |
| MPS Treated | 3/4 (very rare | **1/4 (very rare |
| | within sinusoids | within |
| | and/or portal triads) | leptomeninges) |

Only cells with lymphocyte morphology were considered.
*Values represent the number of animals in which any positive staining was detected over the total number of animals in the group.
**Assessment for presence of lymphocytes could only accurately be performed within leptomeninges given the non-specific staining in the remainder of the tissue.

### Example 4—Additional Constructs can Express Therapeutic Protein

In order to determine if additional constructs comprising a therapeutic gene of interest could deliver a therapeutic protein into a T cell in a site-specific manner, a delivery construct comprising transposon elements and expressing a therapeutic protein was generated.

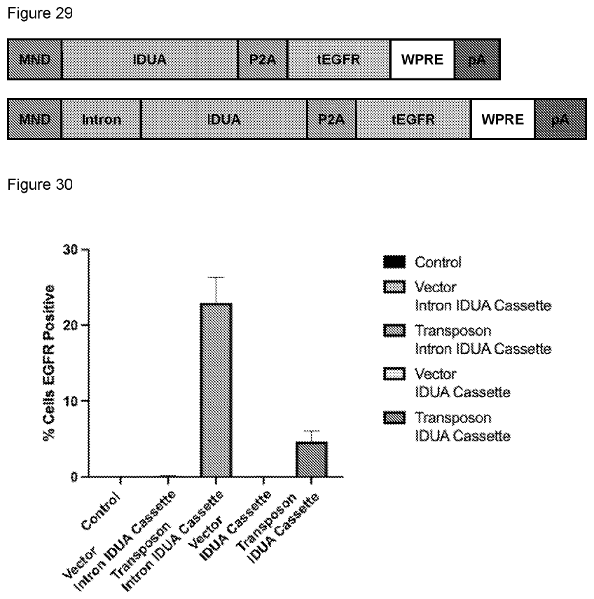

Two IDUA expression plasmids were designed with inverted terminal repeats flanking the gene cassettes for transposon delivery (FIG. 29). One cassette contained uMND-IDUA-P2A-truncated EGFR-WPRE-pA and a second cassette contained uMND-chimeric intron-IDUA-P2A-truncated EGFR-WPRE-pA. After 48 hours of T cell stimulation cells are electroporated with TcBuster™ (R&D Systems, Minneapolis, MN) mRNA and a plasmid cassette containing Transposon ITR elements for integration. Bulk T cells from two separate donors were electroporated with transposase mRNA and DNA IDUA expression cassettes. These cells were then transferred into media and continued to be cultured 96 hours after electroporation human EGFR expression was measured through flow cytometry. Both transposon delivery plasmids had EGFR expression indicating integration of the IDUA cassettes into the genome (FIG. 30).

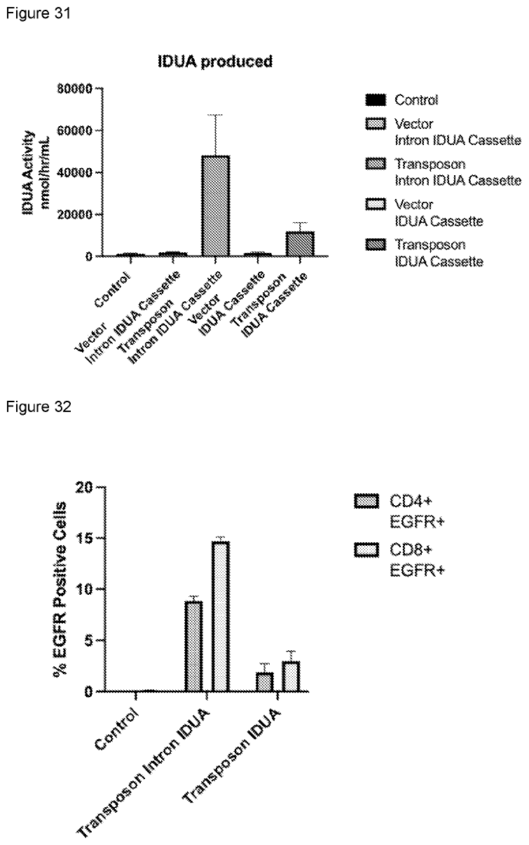

IDUA activity in cell culture media was measured at 96 hours after electroporation with T cell culture media being replaced 24 hours prior to collection. Cell expansion was observed in T cell media and with Dynabead stimulation. The two conditions with the highest IDUA activity had all components for transposon delivery for the IDUA expression cassettes indicating integration into the genome (FIG. 31).

Flow cytometry of the engineered bulk T cells was done 7 days after the cells were engineered. EGFR positive CD4 and CD8 cells were present in the culture (FIG. 32). This indicates that transposon delivery of IDUA expression cassettes can engineer multiple T cell subsets within a bulk population of T cells.

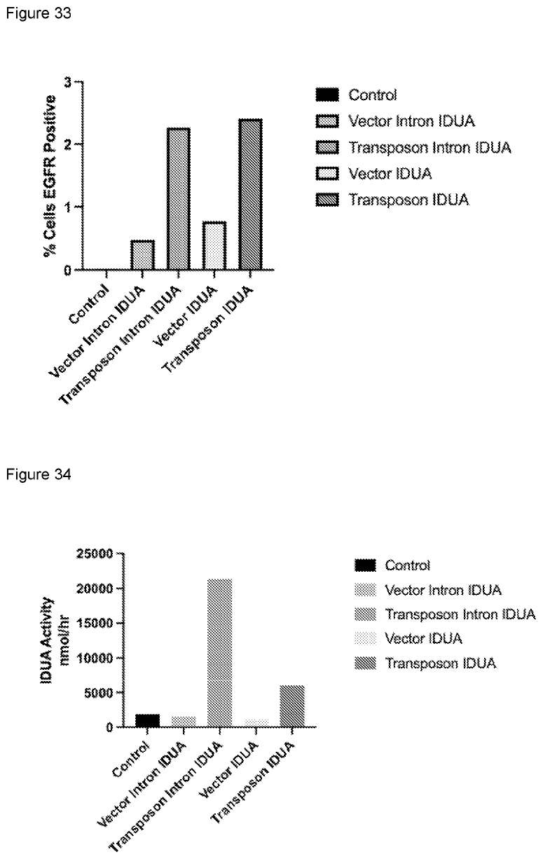

CD8 T cells from P14 mice were electroporated with transposase mRNA and DNA IDUA expression cassettes described above. 96 hours after electroporation murine EGFR expression was measured though flow cytometry. Both transposon delivery plasmids had EGFR expression indicating integration of the IDUA cassettes into the genome (FIG. 33).

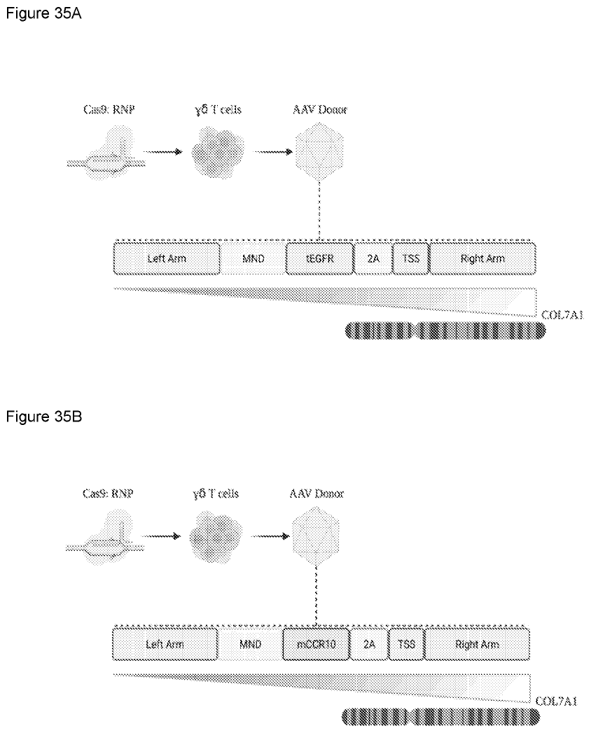

Secreted enzymatic IDUA activity from the Murine P14 CD8 T cells engineered by transposon elements was assessed. IDUA activity in cell culture media was measured at 96 hours after electroporation with T cell culture media being replaced 24 hours prior to collection. Cell expansion was observed in T cell media. The two conditions with the highest IDUA activity had all components for transposon delivery for the IDUA expression cassettes indicating integration into the genome (FIG. 34).

Expression of larger genes may be more difficult using the AAV methods described above. In order to express larger genes of interest, a construct was designed in which a longer polynucleotide was divided and used as the homology arms for the vector. FIG. 35 shows expression constructs comprising an MND promoter followed by tEGFR-T2A-TSS or mCCR10-T2A-TSS coding sequence and flanked by 1000 bp homology arms on both sides targeting the endogenous COL7A1 locus.

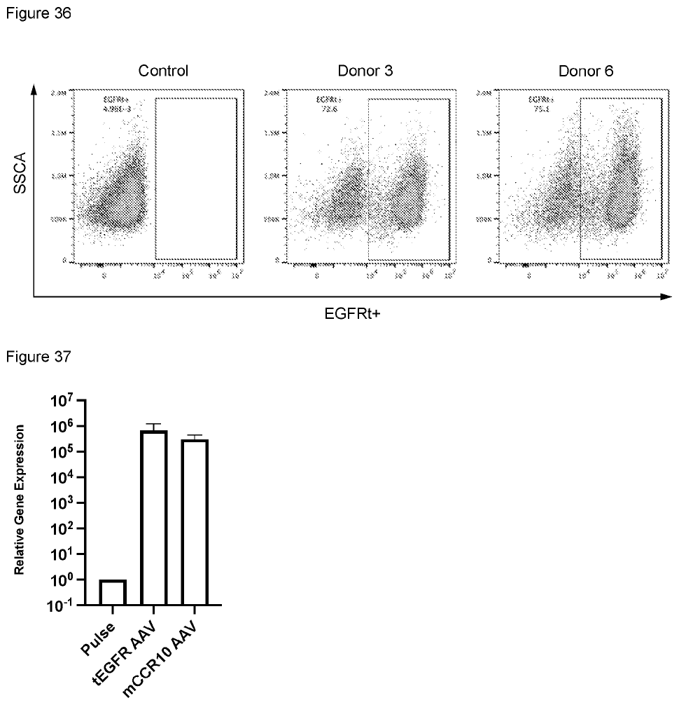

γδ T cells were isolated from PBMCs and stimulated for 2 days before electroporation and AAV6 delivery. The cells were expanded for 9 days before a secondary stimulation and additional 11 day expansion. CAS9 RNP were electroporated into γδ T-cells followed by addition of an HDR template delivered by AAV6 resulting in the coordinated expression of tEGFR or mCCR10 and endogenous COL7A1. CRISPR/Cas9 electroporation with MND-tEGFR-T2A-TSS resulted in efficient integration in γδ T cells. Frequency of successfully targeted γδ T cells as measured by tEGFR expression is shown in FIG. 36.

Next the relative gene expression of COL7A1 from engineered T cells was measured. Edited cells expressed ˜100,000 fold greater amounts of COL7A1 mRNA than the pulse control (FIG. 37). Junction PCR of the 5′ end detected proper integration at COL7A1 using primers inside of the MND promoter and outside of the left homology arm for both tEGFR AAV and mCCR10 AAV.

These results show that larger genes of interest can be used in the expression cassettes described herein the generate edited T cells that express a therapeutic protein.

Numerous modifications and variations in the disclosure as set forth in the above illustrative examples are expected to occur to those skilled in the art. Consequently, only such limitations as appear in the appended claims should be placed on the disclosure.

What is claimed is:

1. A method for genome engineering a T cell or a population of T cells to overexpress a gene of interest comprising introducing into the T cell or T cell population a viral vector, plasmid, nanoplasmid or mini-circle comprising an expression cassette comprising a homology arm (HA), a polynucleotide encoding a gene of interest, a splice acceptor site, a promoter and a targeting site for a nuclease dependent cleavage system targeting molecule,
wherein the expression cassette is inserted at a locus targeted by the targeting molecule.

2. A method for genome engineering a T cell or a population of T cells to overexpress an endogenous gene comprising introducing into the T cell or T cell population a viral vector, plasmid, nanoplasmid or mini-circle comprising an expression cassette comprising a homology arm(s) (HA), a splice acceptor site, a promoter and a targeting site for a nuclease dependent cleavage system targeting molecule,
wherein the expression cassette is inserted upstream of a target gene to be overexpressed.

3. The method of claim 1 or 2, wherein the homology arms are between 35 and 1000 nucleotides.

4. The method of any one of claims 1-3, wherein the nuclease dependent cleavage system comprises a CRISPR/Cas system, a Cas-CLOVER system, a zinc-finger nuclease (ZFN) system, a transcription activator like effector nuclease (TALEN) system, or a meganuclease system.

5. The method of claim 4, wherein the CRISPR/Cas system comprises Cas9, Cas12a, Cas13a or Cas13b.

6. The method of any one of claims 1-5, wherein the nuclease dependent cleavage system is a CRISPR/Cas system and the targeting molecule is a guide RNA.

7. The method of claim 6, wherein the method further comprises transfecting the T cell or population of T cells with a Cas protein or polynucleotide encoding a Cas protein and guide RNA molecules that direct integration of the expression cassette to a target locus in the T cell genome.

8. The method of claim 7 wherein the target locus is an AAVS1 or T-cell receptor a constant (TRAC) locus.

9. The method of any one of claims 1-3, wherein the plasmid comprises inverted terminal repeats flanking the polynucleotide encoding a gene of interest for transposon delivery.

10. The method of claim 8A, wherein the plasmid for transposon delivery comprises a promoter, a gene of interest, a biomarker, a regulatory element and optionally a chimeric intron.

11. The method of any one of the preceding claims, further comprising introducing into the T cell or T cell population a polynucleotide encoding a biomarker molecule useful to enrich for the T cell or a population of T cells.

12. The method of claim 11, wherein the biomarker molecule comprises a fragment of CD34 and a fragment of CD20.

13. The method of claim 11 or 12, wherein the biomarker polynucleotide is on the same expression cassette as the homology arm(s), splice acceptor site, promoter, and targeting site for a nuclease dependent cleavage system targeting molecule.

14. The method of any one of the preceding claims, wherein the viral vector is a lentiviral vector, adenoviral vector, or AAV vector.

15. The method of any one of the preceding claims, wherein the viral vector is selected from the group consisting of a VSVg-pseudotype lentiviral vector, AAV6 vector, AAV1 vector or AAV-DJ vector.

16. The method of any one of the preceding claims, wherein the gene of interest integrates into the T cell genome via homology directed repair (HDR), homology-mediated end joining (HMEJ) or a combination of HDR/HMEJ.

17. The method of any one of the preceding claims, wherein the introduction of the plasmid, nanonplasmid or mini-circle is by transfection or electroporation.

18. The method of any one of the preceding claims, wherein the introduction of the viral vector is by electroporation.

19. The method of claim 18, wherein the viral vector is introduced at a multiplicity of infection of 3×10⁵-1×10⁷.

20. The method of claim 17, wherein a plasmid for transposon delivery is electroporated with transposase mRNA and an expression cassette expressing the gene of interest.

21. The method of any one of the preceding claims, wherein efficiency of introduction is greater than 15%.

22. The method of any one of the preceding claims, wherein the T cell population has a viability of greater than 60% after 3 days.

23. The method of any one of the preceding claims, wherein the T cell or population of T cells is a CD4+ T cell, CD8+ T cell, T cell line, primary T cell, naïve T cell, effector T cell, regulatory T cell, memory T cell, or gamma-delta T cell.

24. The method of any one of the preceding claims, wherein the viral vector, plasmid nanoplasmid or mini-circle comprises the promoter next to or near the gene of interest.

25. The method of any one of the preceding claims, wherein the promoter is an MND promoter, a CMV promoter, a CAG promoter, a PGK promoter, a EF1A promoter, an AAV promoter, or a T cell-specific promoter.

26. The method of any one of the preceding claims, wherein the gene of interest is a therapeutic gene or encodes a therapeutic protein.

27. The method of claim 26, wherein the therapeutic gene encodes an enzyme, a cytokine, a chemokine, a T cell receptor, or a cell surface receptor.

28. The method of any one of the preceding claims, wherein the gene of interest is a donor polynucleotide that corrects a mutated genotype in a subject.

29. The method of any one of the preceding claims, wherein the viral vector, plasmid, nanoplasmid or mini-circle further comprises a polynucleotide encoding a T cell receptor or fragment thereof or a chimeric antigen receptor.

30. A method of making a gene edited T cell or population of T cells, comprising:
i) contacting a T cell or population of T cells with a viral vector, plasmid, nanoplasmid or mini-circle comprising an expression cassette comprising a homology arm (HA), a splice acceptor site, a promoter and a targeting site for a nuclease dependent cleavage system targeting molecule, and optionally comprising a polynucleotide encoding a gene of interest;
ii) culturing the T cell or population of T cells of i) in a media that promotes expansion of T cells;
iii) isolating the T cell or population of T cells of ii) based on identification of a marker expressed only on a T cell or population of T cells carrying the viral vector, plasmid, nanoplasmid or mini-circle;
iv) culturing the isolated cells of iii) in a culture medium to expand the isolated cells expressing the gene of interest.

31. The method of claim 30, further comprising a step of stimulating, proliferating or activating the T cell or population of T cells prior to the contacting step.

32. The method of claim 31, wherein the step of stimulating, proliferating or activating the T cell or population of T cells comprises contacting the cell(s) with one or more of IL-2, IL-7, IL-15, IFN-γ, N-acetyl cysteine (NAC).

33. The method of any one of claims 30-32, wherein the method produces gene edited T cells with an efficiency of greater than 15%.

34. The method of any one of claims 30-33, wherein the method maintains 60% viability of cells in culture after 3 days.

35. A gene edited T cell or population of T cells made by the method of any one of claims 1-34.

36. A gene edited T cell comprising
i) a heterologous polynucleotide sequence encoding a gene of interest integrated in the T cell genome at a target location mediated by a nuclease dependent cleavage system, wherein the heterologous polynucleotide sequence is also flanked by portions of a homology arm and expressed via an endogenous promoter; and
ii) a heterologous biomarker molecule.

37. The gene edited T cell of claim 36, wherein the T cell is a CD4+ T cell, CD8+ T cell, T cell line, primary T cell, naïve T cell, effector T cell, regulatory T cell, memory T cell, or gamma-delta T cell.

38. The gene edited T cell of any one of claim 36 or 37, wherein the gene of interest is a therapeutic gene or encodes a therapeutic protein.

39. The gene edited T cell of claim 38, wherein the therapeutic gene encodes an enzyme, a cytokine, a chemokine, a T cell receptor, or a cell surface receptor.

40. The gene edited T cell of any one of claim 36 or 39, wherein the gene of interest is a donor polynucleotide that corrects a mutated genotype in a subject.

41. A method of treating a disease or condition in a subject in need thereof comprising administering to the subject a gene edited T cell or population of T cells of any one of claims 35-40.

42. The method of claim 41, wherein the disease is an enzymopathy, an infection, or a genetic disorder.

43. The method of claim 41 or 42, wherein the disease is an enzymopathy.

44. The method of claim 42 or 43, wherein the enzymopathy is selected from the group consisting of aspartylglucosaminuria, cholesterol ester storage disease, Wolman disease, metachromatic leukodystrophy, Danon disease, Fabry disease, Farber lipogranulomatosis, Farber disease, fucosidosis, galactosialidosis types I/II, Gaucher disease types I/II/III, globoid cell leukodystrophy, Krabbe disease, glycogen storage disease II, Pompe disease, GM1-gangliosidosis types I/II/III, GM2-gangliosidosis type I, Tay Sachs disease, GM2-gangliosidosis type II, Sandhoff disease, GM2-gangliosidosis, α-mannosidosis types I/II, β-mannosidosis, mucolipidosis type I, sialidosis types I/II, mucolipidosis types II/III, I-cell disease, mucolipidosis type IIIC, pseudo-Hurler polydystrophy, mucopolysaccharidosis type I, mucopolysaccharidosis type II, Hunter syndrome, mucopolysaccharidosis type IIIA, Sanfilippo syndrome type B, Sanfilippo syndrome type C, Sanfilippo syndrome type D, mucopolysaccharidosis type IIIB, mucopolysaccharidosis type IIIC, mucopolysaccharidosis type IIID, mucopolysaccharidosis type IVA, mucopolysaccharidosis type IVB Morquio syndrome type A, Morquio syndrome type B, mucopolysaccharidosis type VI, mucopolysaccharidosis type VII, Sly syndrome, mucopolysaccharidosis type IX, multiple sulfatase deficiency, neuronal ceroid lipofuscinosis, CLN1 Batten disease, CLN2 Batten disease, Niemann-Pick disease types A/B, Niemann-Pick disease, Niemann-Pick disease type C1, Niemann-Pick disease type C2, pycnodysostosis, Schindler disease types I/II, and sialic acid storage disease, hemophilia A, hemophilia B, Christmas disease, and Factor VII deficiency, spinal muscular atrophy, and epidermolysis bullosa dystrophica.

45. The method of claim 44, wherein the enzymopathy is mucopolysaccharidosis type I (MPS I) and the gene of interest is iduronidase.

46. The method of claim 45, wherein the administration ameliorates one or more symptoms of MPS I.

47. The method of claim 46, wherein the one or more symptoms are selected from the group consisting of reduction of glycosaminoglycan (GAG) species in tissues and/or urine, increase of IDUA expression in tissues, improved cognition, and reduced vacuolated endothelial cells/foam cells in tissue.

48. The method of claim 42, wherein the genetic disorder is selected from the group consisting of muscular dystrophy, cystic fibrosis, Sickle cell anemia, β-thalassemia, a lysosomal storage disorder, Adenosine Deaminase Deficiency, Severe Combined Immunodeficiency (SCID), Retinitis Pigmentosa, macular degeneration, and Wiskott-Aldrich Syndrome.

49. The method of any one of claims 42-48, wherein the T cell is a CD4+ T cell, CD8+ T cell, T cell line, primary T cell, naïve T cell, effector T cell, regulatory T cell, memory T cell, or gamma-delta T cells.

50. The method of any one of claims 42-49, wherein the T cell is first isolated from the subject to be treated and then genetically modified according to the method of any one of claims 1-31.

51. A polynucleotide expression cassette comprising a homology arm (HA), a polynucleotide encoding a gene of interest, a splice acceptor site, a promoter and a targeting site for a nuclease dependent cleavage system targeting molecule, wherein the expression cassette is capable of insertion at a locus targeted by the targeting molecule.

52. A polynucleotide expression cassette comprising homology arm(s) (HA), a splice acceptor site, a promoter and a targeting site for a nuclease dependent cleavage system targeting molecule, wherein the expression cassette is capable of insertion upstream of a target gene to be overexpressed.

53. A polynucleotide expression cassette comprising homology arm(s) (HA), a splice acceptor site, a promoter and a targeting site for transposon delivery of a gene of interest, wherein the expression cassette is capable of insertion at a locus targeted by the transposon delivery site and/or is capable of insertion upstream of a target gene to be overexpressed.

54. The expression cassette of claim 52 to 53, wherein the homology arms are between 35 and 1000 nucleotides.

55. The expression cassette of any one of claims 51-54, wherein the nuclease dependent cleavage system comprises a CRISPR/Cas system, a Cas-CLOVER system, a zinc-finger nuclease (ZFN) system, a transcription activator like effector nuclease (TALEN) system, or a meganuclease system.

56. The expression cassette of any one of claims 51-55, wherein the nuclease dependent cleavage system is a CRISPR/Cas system and the targeting molecule is a guide RNA.

57. The expression cassette of any one of claim 46 or 48-50 wherein the locus targeted is an AAVS1 or T-cell receptor a constant (TRAC) locus.

58. The expression cassette of any one of claims 51-57, further comprising a polynucleotide encoding a biomarker molecule useful to enrich for the T cell or a population of T cells.

59. The expression cassette of claim 58, wherein the biomarker molecule comprises a fragment of CD34 and a fragment of CD20.

60. The expression cassette of any one of claims 55-59, wherein the expression cassette comprises the promoter next to or near the gene of interest.

61. The expression cassette of any one of claims 51-60, wherein the promoter is an MND promoter, a CMV promoter, a CAG promoter, a PGK promoter, a EF1A promoter, an AAV promoter, or a T cell-specific promoter.

62. The expression cassette of any one of claims 51-61, wherein the gene of interest is a therapeutic gene or encodes a therapeutic protein.

63. The expression cassette of claim 62, wherein the therapeutic gene encodes an enzyme, a cytokine, a chemokine, a T cell receptor, or a cell surface receptor.

64. The expression cassette of claim 63, wherein the gene of interest is a donor polynucleotide that corrects a mutated genotype in a subject.

65. The expression cassette of any one of claims 51-64, wherein the expression cassette further comprises a polynucleotide encoding a T cell receptor or fragment thereof or a chimeric antigen receptor.

66. A viral vector, plasmid, nanoplasmid or mini-circle comprising an expression cassette of any one of claims 51-65.

67. The viral vector of claim 66, wherein the viral vector is a lentiviral vector, adenoviral vector, or AAV vector.

68. The viral vector of claim 67, wherein the viral vector is selected from the group consisting of a VSVg-pseudotype lentiviral vector, AAV6 vector, AAV1 vector or AAV-DJ vector.
